SEQUENCE LISTING (14 sequences)

SEQ ID NO: 1 - protein, 5 aa, synthetic construct
  REGION 1..5: note = Synthetic
SYAMS

SEQ ID NO: 2 - protein, 17 aa, synthetic construct
  REGION 1..17: note = Synthetic
GISAIDQSTYYADSVKG

SEQ ID NO: 3 - protein, 17 aa, synthetic construct
  REGION 1..17: note = Synthetic
QKFMQLWGGGLRYPFGY

SEQ ID NO: 4 - protein, 126 aa, synthetic construct
  REGION 1..126: note = Synthetic
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 5 - protein, 11 aa, synthetic construct
  REGION 1..11: note = Synthetic
SGEGMGDKYAA

SEQ ID NO: 6 - protein, 7 aa, synthetic construct
  REGION 1..7: note = Synthetic
RDTKRPS

SEQ ID NO: 7 - protein, 9 aa, synthetic construct
  REGION 1..9: note = Synthetic
GVIQDNTGV

SEQ ID NO: 8 - protein, 106 aa, synthetic construct
  REGION 1..106: note = Synthetic
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 9 - protein, 212 aa, synthetic construct
  REGION 1..212: note = Synthetic
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 456 aa, synthetic construct
  REGION 1..456: note = Synthetic
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 270 aa, synthetic construct
  REGION 1..270: note = Synthetic
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 12 - protein, 34 aa, synthetic construct
  REGION 1..34: note = Synthetic
MHHHHHHHHHHAAGLNDIFEAQKIEWHEAAIEGR

SEQ ID NO: 13 - protein, 159 aa, synthetic construct
  REGION 1..159: note = Synthetic
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 14 - protein, 159 aa, synthetic construct
  REGION 1..159: note = Synthetic
SITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

(19) United States
(12) Patent Application Publication
(10) Pub. No.: US 2025/0215074 A1
(43) Pub. Date: Jul. 3, 2025

(54) METHODS FOR REDUCING RESPIRATORY INFECTIONS

(71) Applicant: MEDIMMUNE LIMITED, Cambridge (GB)

(72) Inventors: Emma Suzanne COHEN, Cambridge (GB); Xavier ROMERO ROS, Cambridge (GB); Sam STRICKSON, Cambridge (GB); Victor AUGUSTI NEGRI, Cambridge (GB)

(21) Appl. No.: 18/850,285

(22) PCT Filed: Mar. 24, 2023
(86) PCT No.: PCT/EP2023/057583

§ 371 Date: Sep. 24, 2024

Related U.S. Application Data
(60) Provisional application No. 63/323,742, filed on Mar. 25, 2022.

(51) Int. Cl. C07K 16/24

(52) U.S. Cl. CPC C07K 16/244; C07K 2317/565; C07K 2317/76; C07K 2319/21; C07K 2319/50

(57) ABSTRACT

The disclosure relates to methods for reducing infection, particularly respiratory viral infections, for example, in subjects with COPD. The methods comprise the use of IL-33 antagonists, particularly oxIL-33 antagonists.

### CROSS-REFERENCE TO RELATED APPLICATIONS

This application claims benefit under 35 U.S.C. § 119 (e) of U.S. Provisional Patent Application No. 63/323,742, filed Mar. 25, 2022, which is incorporated by reference herein in its entirety for all purposes.

### REFERENCE TO SEQUENCE LISTING SUBMITTED ELECTRONICALLY

This application incorporates by reference a Sequence Listing submitted with this application in computer readable form (CRF) as a text file entitled “IL33-207-US-PSP-SequenceListing” created on Feb. 17, 2023 and having a size of 19,729 bytes.

### TECHNICAL FIELD

The disclosure relates to methods for reducing or preventing infection, particularly respiratory tract viral infections (RTVIs), in subjects with IL-33-mediated respiratory disorders, for example, in subjects with COPD.

### BACKGROUND

Exacerbations of chronic obstructive lung disease (COPD) are a source of significant suffering for patients and a major cause of morbidity and mortality and hospital admissions. Respiratory tract infections, particularly viral infections, seem to be implicated in approximately half of all COPD exacerbations. Only a quarter of exacerbations appear unrelated to infection.

Prevention of COPD exacerbations is an important part of the management of COPD. Accordingly. strategies to prevent infections that lead to exacerbations are desired. They may have a significant effect on morbidity of COPD as well as improve quality of life in subjects with COPD.

### SUMMARY OF THE DISCLOSURE

Subjects with COPD are particularly susceptible to airway infections that lead to acute exacerbations of COPD. Club cells are an important secretory cell type of the respiratory epithelium with a variety of cell defence functions. Inhibition of club cell activity has been directly implicated in increasing susceptibility of the airway epithelium to infections, such as respiratory syncytial viral (RSV) infection. RSV infection is one of a number of respiratory tract viral infections (RTVI) known to lead to acute exacerbation events in COPD (AECOPD)(Wedzicha Proc Am Thorac Soc Vol 1. pp 115-120, 2004). This disclosure relates to the discovery that the oxidised form of IL-33 (IL-33ox) attenuates club cell activity in COPD epithelia. The examples show that blocking IL-33ox activity directly repairs club cell activity in COPD air liquid interface (ALI) cultures. The disclosure therefore suggests that blocking oxIL-33 activity in subjects with COPD can enhance club cell activity to reduce infections that lead to acute exacerbations of COPD. Such methods in a clinical setting may have a significant impact on morbidity of COPD and improve quality of life.

Accordingly, in one aspect, the disclosure provides an IL-33 antagonist for use in a method of treatment reducing or preventing respiratory tract infection in a subject with an IL-33-mediated respiratory disorder. In some instances, the IL-33-mediated respiratory disorder is chronic obstructive pulmonary disease (COPD). In some instances, the infection is a respiratory tract viral infection or a respiratory tract bacterial infection.

In some instances, the infection is a respiratory tract viral infection (RTVI). In some instances, the respiratory tract viral infection caused by an influenza virus (e.g., Influenza virus A, Influenza virus B), respiratory syncytial virus (RSV), adenovirus, metapneumovirus, cytomegalovirus, parainfluenza virus (e.g., hPIV-1, hPIV-2, hPIV-3, hPIV-4), rhinovirus, adenovirus, coxsackie virus, echo virus, corona virus, herpes simplex virus, SARS-coronavirus or smallpox.

In some instances, the IL-33 antagonist inhibits IL-33ox activity, thereby increasing club cell activity in the airway epithelium.

In some instances, the IL-33 antagonist inhibits IL-33ox activity, thereby increasing total club cell area in the airway epithelium.

In some instances, the IL-33 antagonist inhibits oxIL-33 activity, thereby increasing mRNA expression levels in the airway epithelium of one or more markers selected from: SCGBIBA1, BPIFA1. SCGB3A1, WFDC2, MSMB, LTF, SLPI, C3, HLA-DRA, CXCL1, CD74, CXCL17, MDK, TGM2, HLA-DRB1, CXCL8, CXCL2, HLA-DRB5, CX3CL1 and HLA-DPA1. In some instances, the one or more markers is selected from: SCGBIBA1, BPIFA1, SCGB3A1, WFDC2, MSMB and LTF. In some instances, the one or more markers comprise SCGB1BA1 and/or BPIFA1.

In some instances, the IL-33 antagonist inhibits oxIL-33 activity, thereby increasing protein expression levels in the airway epithelium of one or more markers selected from: CCSP, SCGB3A1, WFDC2, Beta-microseminoprotein, lactotransferrin, SPLUNC1, secretory leukocyte protease inhibitor (SLPI), Complement C3, HLA-DR alpha chain, C—X—C motif chemokine ligand 1 (CXCL1), Cluster of Differentiation 74 (CD74), C—X—C motif chemokine 17 (CXCL17), midkine (MDK), Protein-glutamine gamma-glutamyltransferase 2 (TGM2), HLA class II histocompatibility antigen, DRB1 beta chain (HLA-DRB1), chemokine (C—X—C motif) ligand 8 (CXCL8), Chemokine (C—X—C motif) ligand 2 (CXCL2), HLA class II histocompatibility antigen, DRB5 beta chain (HLA-DRB5), chemokine (C-X3-C motif) ligand 1 (CX3CL1) and Major histocompatibility complex, class II, DP alpha 1 (HLA-DPA1). In some instances, the one or more markers are selected from: CCSP, SCGB3A1, WFDC2, Beta-microseminoprotein, lactotransferrin and SPLUNC1. In some instances, the one or more markers comprise CCSP and/or SPLUNC1.

In some instances, the airway epithelium comprises the lower airway epithelium, such as cuboidal epithelium or squamous epithelium. In some instances, the airway epithelium comprises the upper airway epithelium, such as ciliated pseudostratified columnar epithelium.

In some instances, IL-33 antagonist inhibits IL-33ox activity, thereby increasing club cell defence function in the airway epithelium. In some instances, increasing club cell defence function in the airway epithelium comprises increasing the activity of one or more proteins selected from: CCSP, SCGB3A1, WFDC2, Beta-microseminoprotein, lactotransferrin, SPLUNC1, SLPI, C3, HLA-DRA, CXCL1, CD74, CXCL17, MDK, TGM2, HLA-DRB1, CXCL8, CXCL2, HLA-DRB5, CX3CL1 and HLA-DPA1. These proteins have been implicated in epithelial defence functions. Therefore, increasing their expression by inhibiting oxIL-33 activity is likely to improve resistance to respiratory infection.

In some instances, the method reduces the annualised exacerbation rate in the subject. In some instances, the method reduces the frequency of acute exacerbations of COPD (AECOPD) in the subject.

In some instances, the IL-33 antagonist is an IL-33ox antagonist.

In some instances, the antagonist is an antibody or antigen binding fragment thereof. In some instances, the antibody or antigen binding fragment thereof binds specifically to the reduced form of IL-33 (redIL-33).

In some instances, the anti-IL-33 antibody or antigen binding fragment thereof comprises a VH domain comprising HCDR1 having the sequence set forth in SEQ ID NO: 1: HCDR2 having the sequence set forth in SEQ ID NO: 2; and HCDR3 having the sequence set forth in SEQ ID NO: 3; and a VL domain comprising LCDR1 having the sequence set forth in SEQ ID NO: 5: LCDR2 having the sequence set forth in SEQ ID NO: 6 and LCDR3 having the sequence set forth in SEQ ID NO: 7. In some instances, the anti-IL-33 antibody is tozorakimab.

In another aspect, the disclosure provides a composition comprising the IL-33 antagonist disclosed herein for use in a method of treatment disclosed herein.

In another aspect, the disclosure provides a method of treatment reducing or preventing respiratory infection in a subject with an IL-33-mediated respiratory disorder comprising administering to the subject a therapeutically effective amount of an IL-33 antagonist.

In another aspect, the disclosure provides the use of an IL-33 antagonist for use in the manufacture of a medicament for a treatment reducing or preventing respiratory infections in a subject with an IL-33-mediated respiratory disorder.

In another aspect, the disclosure provides an IL-33 antagonist for use in reducing AECOPD in a subject with COPD, wherein the IL-33 antagonist inhibits IL-33ox activity, thereby reducing respiratory tract infections and AECOPD in the subject.

### BRIEF DESCRIPTION OF DRAWINGS

FIG. 1 Schematic representation of ALI cultures and endpoint assays

FIG. 2 Volcano plot representing differential expression of genes from bulk RNA sequencing in ALI cultures treated with IL-33°N versus untreated control

FIG. 3 Visual representation of changes in the proportion of cell types in ALI cultures after treatment with IL-33^{ox} compared with untreated control

FIG. 4 Heat map showing the scale-normalized average expression levels of genes associated with mucin production or defence in the secretory states in ALI cultures treated with IL-33°N or untreated control

FIG. 5 Representative immunohistochemistry of COPD ALI cultures following treatment with IL-33-neutralizing antibody (tozorakimab) or hIgG1 isotype control antibody. MUC5AC/AB for goblet cells (yellow), acetylated α-tubulin for ciliated cells (teal) and p63 for basal cells (purple)(scale bar=70 μm)

FIG. 6 Flow cytometry analysis of intracellular MUC5AC to quantify the proportion of MUC5AC single-positive goblet cells in dissociated healthy or COPD ALI cultures following treatment with tozorakimab, ST2-neutralizing or the relevant isotype control antibodies, or untreated control (n=6 healthy, n=5 COPD)

FIG. 7 ELISA of MUC5AC secreted into the apical region of healthy or COPD ALI cultures incubated with tozorakimab. ST2-neutralizing or the relevant isotype controls, or untreated control (n=7 healthy, n=6 COPD)

FIG. 8 Volcano plot representing differential expression of genes from bulk RNA sequencing in COPD ALI cultures treated with IL-33-neutralizing antibody (tozorakimab)

FIG. 9 Heat map showing changes in gene expression levels in COPD ALI cultures following treatment with hIgG1 isotype control antibody or tozorakimab by gene families

FIG. 10A Heat map showing the scale-normalized average expression levels of genes associated with mucin production or defence in the secretory states in COPD ALI cultures treated with tozorakimab (MEDI3506) or untreated control

FIG. 10B Heat map showing the scale-normalized average expression levels of additional genes associated with defence in the secretory states in COPD ALI cultures treated with tozorakimab (MEDI3506) or untreated control

### DISCLOSURE OF THE INVENTION

### General Definitions

“Abnormal” as employed herein means a difference in a function compared with said function in a healthy subject, typically an increase or a decrease in a function compared with said function in a healthy subject.

“Abnormal epithelium physiology” as employed herein means any abnormality in the functioning of an epithelium in the human body. Functions of epithelium in the human body include: acting as a barrier to protect tissues beneath: regulation and exchange of chemical entities between tissues and a cavity: secretion of chemicals into a cavity; and sensation. Abnormalities in any of these functions can have devastating physiological effects. Epithelium is present in a wide range of tissues in the body including the skin, respiratory tract, gastrointestinal tract, reproductive tract, urinary tract, exocrine and endocrine glands, as such, abnormalities within the epithelium can be involved in a wide range of diseases or conditions. In some instances, the epithelium is the airway epithelium and abnormal epithelium physiology is abnormal airway epithelium physiology.

“Antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Antigen binding fragment” and “binding fragment” refers to a molecule other than an intact antibody that comprises a portion of the intact antibody that binds to the antigen to which the intact antibody binds. Examples of antibody fragments include, but are not limited to, Fv, Fab, Fab′, F(ab′)₂, Fab′-SH, diabodies, triabodies, tetrabodies, linear antibodies, single-chain antibody molecules (e.g., scFv), and multispecific antibodies formed from antigen binding fragments. For a review of certain antibody fragments, see Hudson et al. Nat. Med. 9: 129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994): see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097: WO 1993/01161: Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003). Single-domain antibodies are antibody fragments comprising all or a portion of the heavy chain variable domain or all or a portion of the light chain variable domain of an antibody. Antibody fragments can be made by various techniques, including but not limited to proteolytic digestion of an intact antibody as well as production by recombinant host cells (e.g. E. coli or phage).

“Club cells”, also known as bronchiolar exocrine cells and formerly known as Clara cells, are low columnar/cuboidal cells with short microvilli, predominantly found in the small airways (bronchioles) of the lungs. Club cells are found in the ciliated simple epithelium. One of the main functions of club cells is to protect the bronchiolar epithelium, which they do, for example, by secreting club cell secretory protein (CCSP, also known as uteroglobin, CC10 or CC16: UniProtKB accession number P11684). CCSP has putative anti-inflammatory functions that have been strongly implicated in modulating inflammatory responses to infection. Wang et al demonstrate that CCSP null mice infected with RSV increased viral persistence, lung inflammation and airway reactivity (Wang et al The Journal of Immunology, 2003, 171:1051-1060). Therefore, increasing expression of CCSP in the small airways may reduce or prevent viral infections such as RSV infection. CCSP is encoded by the gene SCGB1A1.

“IL-33” protein as employed herein refers to interleukin 33, in particular a mammalian interleukin 33 protein, for example human protein deposited with UniProt number 095760. IL-33 is not a single species but exists as reduced and oxidized forms. The reduced form of IL-33 undergoes rapid oxidation in vivo, for example in the timeframe of 5 minutes to 40 minutes. The terms “IL-33” and “IL-33 polypeptide” are used interchangeably. In certain instances, IL-33 is full length. In another instances, IL-33 is mature, truncated IL-33 (amino acids 112-270). Recent studies suggest full length IL-33 is active (Cayrol and Girard, Proc Natl Acad Sci USA 106 (22): 9021-6 (2009); Hayakawa et al., Biochem Biophys Res Commun. 387 (1): 218-22 (2009): Talabot-Ayer et al, J Biol Chem. 284 (29): 19420-6 (2009)). However, N-terminally processed or truncated IL-33 including but not limited to aa 72-270, 79-270, 95-270. 99-270, 107-270, 109-270, 111-270, 112-270 may have enhanced activity (Lefrancais 2012, 2014).

“Oxidized IL-33”, “oxIL-33” or “IL-33ox” refers to a form of IL-33 that binds to RAGE, and triggers RAGE-EGFR mediated signalling. It has been previously shown that the activation of the IL-33ox-RAGE/EGFR pathway drives pathogenic changes in lung epithelial composition (as disclosed in WO 2021/089563, which is hereby incorporated by reference in its entirety). Oxidised IL-33 is a protein visible as a distinct band, for example by western blot analysis under non-reducing conditions, in particular with a mass 4 Da less than the corresponding reduced from. In particular, it refers to a protein with one or two disulphide bonds between the cysteines independently selected from cysteines 208, 227, 232 and 259.

“Ox-IL-33/RAGE/EGFR signalling axis” or “oxIL-33 signalling acis” refers to the RAGE/EGFR signalling pathway activated by oxIL-33 binding to the RAGE/EGFR signalling complex at the surface of epithelial cells.

“Reduced IL-33”, “redIL-33” or “IL-33red” as employed herein refers to the form of the IL-33 that binds to ST2 and triggers ST2 mediated signalling. In particular cysteines 208, 227, 232 and 259 of the reduced form are not disulfide bonded.

References to “WT IL-33” or “IL-33” may refer to either the reduced or oxidised forms, or both, unless it is clear from the context within which it is used that one of the forms is meant.

“IL-33 antagonist” refers to a molecule that inhibits the interaction of an IL-33 axis binding partner with one or more of its binding partners. An IL-33 antagonist may be an IL-33red antagonist, IL-33ox antagonist or an antagonist that inhibits both IL-33red and IL-33ox. The disclosure also contemplates the use “oxIL-33 signalling axis antagonists”, which, in addition to IL-33ox antagonists, includes RAGE and EGFR antagonists, the receptors that complex with IL-33ox to mediate oxIL-33 signalling. Consequently, antagonising the activity of RAGE and/or EGFR may also be beneficial towards inhibiting pathological oxIL-33 signalling mechanisms disclosed herein.

“IL-33-mediated disorder” refers to a disease or disorder in which IL-33 has been shown to have a pathological role. In particular for this disclosure, IL-33 mediated disorders of the respiratory tract are envisaged. These may also be referred to IL-33-mediated respiratory disorders. Particular instances relate to IL-33-mediated respiratory disorders characterised by abnormal epithelium physiology. Such disorders include COPD, asthma, COPD overlap syndrome (ACOS), chronic bronchitis, bronchiectasis and emphysema.

“Exacerbation of COPD” or “COPD exacerbation” means an increase in the severity and/or frequency and/or duration of one or more symptoms or indicia of COPD. An “exacerbation of COPD” also includes any deterioration in the respiratory health of a subject that requires and/or is treatable by a therapeutic intervention (such as, e.g., steroid treatment, antibiotic treatment, inhaled corticosteroid treatment, hospitalization, etc.). In some instances, moderate exacerbations are defined as acute exacerbations of COPD (AECOPD) events that require either systemic corticosteroids (such as intramuscular, intravenous or oral) and/or treatment with antibiotics. In some instances, severe exacerbations are defined as AECOPD events requiring hospitalization, emergency medical care visit, or resulting in death. In some instances, the annualized rate of moderate-to-severe acute exacerbations of COPD (AECOPD) includes moderate exacerbations and severe exacerbations.

A “reduction in the frequency” of an exacerbation of COPD means that a subject who has received an IL-33 antagonist as disclosed herein experiences fewer COPD exacerbations (i.e., at least one fewer exacerbation) after treatment than before treatment, or experiences no COPD exacerbations for at least 4 weeks (e.g., 4, 6, 8, 12, 14, or more weeks) following initiation of treatment with an IL-33 antagonist disclosed herein.

“Reducing infection” or “preventing infection” means that a subject who has received an IL-33 antagonist as disclosed herein experiences fewer infections (i.e., at least one less infection) after treatment than before treatment, or experiences no infections for at least 4 weeks (e.g., 4, 6, 8, 12, 14, or more weeks) following initiation of treatment with an IL-33 antagonist disclosed herein. Given that more than 50% of COPD exacerbations are caused by infections like respiratory tract viral infections, a reduction in COPD exacerbations can be an effective proxy for determining reduced infection rate. If infections are reduced it is expected that the number of exacerbations will reduce concurrently.

“Effective amount” or “therapeutically effective amount” of an agent, e.g., a pharmaceutical formulation, refers to an amount effective, at dosages and for periods of time necessary, to achieve the desired therapeutic or prophylactic result.

The term “subject” refers to an animal, human or non-human, to whom treatment according to the methods of the present invention is provided. Veterinary and nonveterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs. The preferred subject is a human.

### Therapeutic Methods

The present disclosure provides methods for reducing or preventing infection, particularly respiratory tract infection, such as respiratory tract viral infection or respiratory tract bacterial infection. The methods are particularly useful in subjects with IL-33 mediated respiratory disorders, particularly in subjects with abnormal epithelium physiology. Abnormal epithelium physiology may be characterised by an imbalance in the cell types that typically make up the respiratory epithelium.

The disclosure provides evidence that the oxidised form of IL-33 (IL-33ox) inhibits club cell activity in the airway epithelium. Club cells are a secretory epithelial cell type with multiple cell defence functions. Inhibition of IL-33ox activity restores club cell activity to the epithelium, including increasing the expression of club cell-related genes with defence functions. This is likely to improve epithelial defence against infections, such as viral or bacterial infections, that lead to exacerbation events in diseases such as COPD.

Accordingly, in some instances, the disclosure provides an IL-33 antagonist for use, therapeutic methods comprising administration of said IL-33 antagonist, and the use of said IL-33 antagonist in the manufacture of a medicament for, reducing or preventing respiratory infection in subjects with IL-33-mediated respiratory disorders. It is to be understood that for each instance disclosing “an IL-33 antagonist for use”, the corresponding “method of treatment” or “use” of said IL-33 antagonist is envisaged.

In some instances, the IL-33-mediated respiratory disorder is selected from asthma, chronic obstructive pulmonary disease (COPD), asthma COPD overlap syndrome (ACOS), chronic bronchitis or emphysema. In some instances, the IL-33-mediated disorder is COPD. These disorders can manifest abnormal epithelium physiology, in which club cell activity may be reduced. For example, the examples show that air-liquid interface (ALI) cultures of COPD epithelia exhibit reduced total club cell area and reduced mRNA and protein expression levels of club cell markers compared to healthy control ALI. The examples demonstrate that this dysfunction is mediated at least in part by IL-33ox. Total club cell area and club cell marker mRNA and protein expression levels are restored upon treatment with an IL-33 antagonist that inhibits the activity of IL-33ox. Accordingly, the examples show that IL-33 antagonists may be useful for restoring club cell activity in the airway epithelium of subjects with IL-33-mediated respiratory disorders, such as COPD.

In some instances, the epithelium is selected from: squamous, cuboidal, columnar and pseudostratified. In some instances the epithelium is ciliated pseudostratified columnar epithelium. In some instances, the epithelium is cuboidal epithelium. In some instances, the epithelium is squamous epithelium.

In some instances the IL-33-mediated respiratory disorder is COPD. COPD is a chronic inflammatory lung disease that causes obstructed airflow from the lungs. Multiple evidence implicates IL-33 as a driver of chronic inflammation observed in the lungs of COPD subjects. Several clinical trials ongoing with IL-33 antagonists attempt to limit chronic inflammation observed in COPD subjects. However, it was hitherto unknown that IL-33 directly impacts club cell function in the epithelium. Thus, the current disclosure is the first to identify that IL-33 antagonists can be used to directly impact airway epithelium physiology to increase club cell function, thereby improving defence functions against respiratory tract infections.

In some instances, the subject is identified as having “mild.” “moderate.” “severe.” or “very severe” COPD if the subject receives such a diagnosis from a physician, based on the Global Initiative for Chronic Obstructive Lung Disease (GOLD)(Global strategy for the diagnosis, management, and prevention of chronic obstructive pulmonary disease (2017 report)(Available from the website: goldcopd.org/wp-content/uploads/2016/12/wms-GOLD-2017-Pocket-Guide.pdf.)). In these instances, a subject's COPD is classified based on airway limitation severity as tested using post-bronchodilator FEV1. A subject's COPD is classified as “mild” using the GOLD classification system if the subject's FEV1is greater than or equal 25 to 80% of the predicted FEV1. A predicted value for FEV1is based on the FEV1value for an average person of similar age, race, height, and gender with healthy lungs. A subject's COPD is classified as “moderate” on the GOLD classification system if the subject's FEV1is greater than or equal to 50% of the predicted FEV1but less than 80% of the predicted FEV1. A subject's COPD is classified as “severe” on the GOLD classification system if the subject's FEV1is greater than or equal to 30% of the predicted FEV, but less than 50% of the predicted FEV1. A subject's COPD is classified as “very severe” on the GOLD classification system if the subject's FEV 1 is less than 30% of the predicted FEV1.

In some instances, the IL-33 antagonist is for use in preventing or reducing respiratory tract infections. As used herein, “respiratory infection”, “respiratory tract infection” and “RTI” have the same meaning. An RTI is an infection of parts of the body involved in breathing, such as the sinuses, throat, airways or lungs.

In some instances, the IL-33 antagonist is for use in reducing or preventing respiratory infections in the lungs (also termed herein “lung respiratory infections”).

In some instances, the IL-33 antagonist is for use in reducing or preventing respiratory infections in the airways (also termed herein “airway respiratory infections”).

In some instances, the IL-33 antagonist is for use in reducing or preventing respiratory infections in the small airways (also termed herein “small airway respiratory infections”).

Club cells are predominantly located in the bronchioles of the lung, hence the IL-33 antagonist disclosed herein may be particularly beneficial for reducing infections that manifest at sites where club cells are predominantly located.

In some instances, the IL-33 antagonist may be for use in reducing or preventing infections caused by viruses (also termed “respiratory viral infections”, “respiratory tract viral infections”, or “RTVIs”).

In some instances, the IL-33 antagonist may be for use in reducing or preventing RTVIs caused by an influenza virus (e.g., Influenza virus A, Influenza virus B), respiratory syncytial virus (RSV), adenovirus, metapneumovirus, cytomegalovirus, parainfluenza virus (e.g., hPIV-1, hPIV-2, hPIV-3, hPIV-4), rhinovirus, adenovirus, coxsackie virus, echo virus, corona virus, herpes simplex virus, SARS-coronavirus, or smallpox.

In some instances, the IL-33 antagonist may be for use in reducing infections caused by bacteria (also termed “respiratory bacterial infections” or “respiratory tract bacterial infections”). In some instances, the IL-33 antagonist may be for use in reducing infections caused by Chlamydia pneumoniae or Mycoplasma pnuemoniae.

In some instances, reducing or preventing infection comprises increasing total club cell area in the respiratory epithelium. In some instances, the IL-33 antagonist inhibits or reduces IL-33ox activity, thereby increasing total club cell area in the respiratory epithelium. In some instances, the respiratory epithelium is the upper airway epithelium. In some instances, the upper airway epithelium is ciliated pseudostratified columnar epithelium. In some instances, the epithelium is the lower airway epithelium. In some instances, the lower airway epithelium is cuboidal epithelium. In some instances, the lower airway epithelium is squamous epithelium.

The examples show that blocking IL-33ox activity increases the proportion of club cells in COPD ALI cultures, restoring total club cell area similar to that seen in healthy controls. Restoring club cell area increases the expression of club cell defence genes. It follows that increasing club cell area and club cell defence function is likely to reduce or prevent infections in subjects with IL-33-mediated disorders previously prone to RTVIs.

Total club cell area may be measured by measuring markers from a relevant biological sample obtained from the subject. In some instances, the biological sample may be a biopsy, for example, a respiratory epithelium biopsy, bronchial brushing, bronchoalveolar fluid (BALF), sputum, serum, plasma or nasal mucosal lining fluid. In some instances, the biological sample is obtained from the respiratory epithelium. If an increase in the concentration of markers in detected in the subject following treatment, this indicates that the treatment has successfully increased the total club cell area in the respiratory epithelium.

In some instances, the marker may be the mRNA expression level of SCGB1A1. In some instances, the marker may be the mRNA expression level of SCGB3A1. In some instances, the marker may be the mRNA expression level of WFDC2. In some instances, the marker may be the mRNA expression level of MSMB. In some instances, the marker may be the mRNA expression level of BP1FA1. In some instances, an increase of mRNA expression levels of one or more of SCGB1A1, SCGB3A1, WEDC2, MSMB and BPIFA1 following treatment compared to a reference expression level for one or both markers, indicates that the total club cell area has increased.

In some instances, the marker may be the protein expression level of CCSP. In some instances, the marker may be the protein expression level of SPLUNC1. In some instances, the marker may be the protein expression level of Secretoglobin family 3A member 1 (SCGB3A1). In some instances, the marker may be the protein expression level of WAP four-disulfide core domain protein 2 (WFDC2). In some instances, the marker may be the protein expression level of Beta-microseminoprotein. In some instances, an increase of protein expression levels of one or more of CCSP, SCGB3A1, WFDC2, Beta-microseminoprotein, lactotransferrin and SPLUNC1 following treatment compared to a reference expression level for one or both markers, indicates that the total club cell area has increased.

In some instances, the reference expression level is the level determined in a biological sample obtained from the subject prior to treatment with an IL-33 antagonist. In some instances, the mRNA expression level is measured by qRT-PCR. In some instances, the protein expression level is measured by enzyme-linked immunosorbent assay (ELISA), immunohistochemistry (IHC), immunofluorescence, flow cytometry, or Western blot

In some instances, reducing or preventing infection comprises increasing mRNA expression levels of SCGB1A1. In some instances, the IL-33 antagonist inhibits or reduces IL-33ox activity, thereby increasing mRNA expression levels of SCGB1A1. In some instances, the increased mRNA expression level is in the epithelium. In some instances, the increased expression is in the airway epithelium. In some instances, the increased expression is in the upper airway epithelium. In some instances, the increased expression is in ciliated pseudostratified columnar epithelium. In some instances, the increased expression is in the lower airway epithelium. In some instances, the increased expression is in the small airway epithelium. In some instances, the increased mRNA expression level is in cuboidal epithelium. In some instances, the increased mRNA expression level is in squamous epithelium. SCGB1A1 encodes CCSP, which as described elsewhere herein is secreted by club cells and has been shown to regulate lung inflammatory and immune responses to RSV infection (Wang et al The Journal of Immunology, 2003, 171:1051-1060). The examples show that treatment with an IL-33 antagonist increases expression of SCGB1A1 from COPD epithelia, thereby increasing anti-inflammatory and immune response activity to agents that cause infection. In some instances, the increased expression is in club 1 cells, club 2 cells, club 3 cells or club 4 cells.

Suitable samples and methods of measuring and determining an increase in mRNA expression levels of SCGB1A1 are disclosed elsewhere herein.

In some instances, reducing or preventing infection comprises increasing protein expression levels of CCSP. In some instances, the IL-33 antagonist inhibits or reduces IL-33ox activity, thereby increasing protein expression levels of CCSP. In some instances, the increased protein expression level is in the epithelium. In some instances, the increased expression is in the airway epithelium. In some instances, the increased protein expression level is in the upper airway epithelium. In some instances, the increased protein expression level is in ciliated pseudostratified columnar epithelium. In some instances, the increased protein expression level is in the lower airway epithelium. In some instances, the increased protein expression level is in the small airway epithelium. In some instances, the increased protein expression level is in cuboidal epithelium. In some instances, the increased protein expression level is in squamous epithelium.

Suitable samples and methods of measuring and determining an increase in protein expression levels of CCSP are disclosed elsewhere herein.

In some instances, reducing or preventing infections comprises increasing CCSP activity. In some instances, the IL-33 antagonist inhibits or reduces IL-33ox activity, thereby increasing CCSP activity. In some instances, the increased CCSP activity is in the epithelium. In some instances, the increased CCSP activity is in the airway epithelium. In some instances, the increased CCSP activity is in the upper airway epithelium. In some instances, the increased CCSP activity is in ciliated pseudostratified columnar epithelium. In some instances, the increased CCSP activity is in the lower airway epithelium.

In some instances, the increased CCSP activity is in the small airway epithelium. In some instances, the increased CCSP activity is in cuboidal epithelium. In some instances, the increased CCSP activity is in squamous epithelium.

In some instances, reducing or preventing infection comprises increasing mRNA expression levels of BPIFA1. In some instances, the IL-33 antagonist inhibits or reduces IL-33ox activity, thereby increasing mRNA expression levels of BP1FA1. In some instances, the increased mRNA expression level is in the epithelium. In some instances, the increased expression is in the airway epithelium. In some instances, the increased expression is in the upper airway epithelium. In some instances, the increased expression is in ciliated pseudostratified columnar epithelium. In some instances, the increased expression is in the lower airway epithelium. In some instances, the increased expression is in the small airway epithelium. In some instances, the increased mRNA expression level is in cuboidal epithelium. In some instances, the increased mRNA expression level is in squamous epithelium. BPIFA1 encodes BPI fold-containing family A member 1 (BPIFA 1, also known as SPLUNC1), which has be shown to play a role in innate immune response in the upper airways. Sayyed et al show that BPIFIA1 protects a host from Pseudomonas aeruginosa bacterial infection in the upper respiratory tract (Sayeed et al Infect. Immun. 81:285-291 (2013)). In some instances, the increased expression is in club 1 cells, club 2 cells, club 3 cells or club 4 cells.

Suitable samples and methods of measuring and determining an increase in mRNA expression levels of BPF1A are disclosed elsewhere herein.

In some instances, reducing or preventing infection comprises increasing protein expression levels of SPLUNC1. In some instances, the IL-33 antagonist inhibits or reduces IL-33ox activity, thereby increasing protein expression levels of SPLUNC1. In some instances, the increased protein expression level is in the epithelium. In some instances, the increased expression is in the airway epithelium. In some instances, the increased protein expression level is in the upper airway epithelium. In some instances, the increased protein expression level is in ciliated pseudostratified columnar epithelium. In some instances, the increased protein expression level is in the lower airway epithelium. In some instances, the increased protein expression level is in the small airway epithelium. In some instances, the increased protein expression level is in cuboidal epithelium. In some instances, the increased protein expression level is in squamous epithelium.

Suitable samples and methods of measuring and determining an increase in protein expression levels of SPLUNC1 are disclosed elsewhere herein.

In some instances, reducing or preventing infections comprises increasing SPLUNC1 activity. In some instances, the IL-33 antagonist inhibits or reduces IL-33ox activity, thereby increasing SPLUNC1 activity. In some instances, the increased SPLUNC1 activity is in the epithelium. In some instances, the increased SPLUNC1 activity is in the airway epithelium. In some instances, the increased SPLUNC1 activity is in the upper airway epithelium. In some instances, the increased SPLUNC1 activity is in ciliated pseudostratified columnar epithelium. In some instances, the increased SPLUNC1 activity is in the lower airway epithelium. In some instances, the increased SPLUNC1 activity is in the small airway epithelium. In some instances, the increased SPLUNC1 activity is in cuboidal epithelium. some instances, the increased SPLUNC1 activity is in squamous epithelium.

In some instances, reducing or preventing infection comprises increasing mRNA expression levels of SCGB3A1. In some instances, the IL-33 antagonist inhibits or reduces IL-33ox activity, thereby increasing mRNA expression levels of SCGB3A1. In some instances, the increased mRNA expression levels is in the epithelium. In some instances, the increased expression is in the airway epithelium. In some instances, the increased expression is in the upper airway epithelium. In some instances, the increased expression is in ciliated pseudostratified columnar epithelium. In some instances, the increased expression is in the lower airway epithelium. In some instances, the increased expression is in the small airway epithelium. In some instances, the increased expression is in the cuboidal epithelium. In some instances, the increased expression is in the squamous epithelium. SCGB3A1 encodes SCGB3A1, which is a cytokine-like protein secreted by club cells that has been shown to inhibit cell growth in vitro (Krop et al PNAS, 2001, 98:9796-9801: Zuo et al Am J Respir Crit Care Med. 2018, 198:1375-1388). The examples show that treatment with an IL-33 antagonist increases expression of SCGB3A1 from COPD epithelia. In some instances, the increased expression is in club 1 cells, club 2 cells, club 3 cells or club 4 cells. In some instances, the increased expression is in club 1, club 2 or club 3 cells.

Suitable samples and methods of measuring and determining an increase in mRNA expression levels of SCGB3A1 are disclosed elsewhere herein.

In some instances, reducing or preventing infection comprises increasing protein expression levels of SCGB3A1. In some instances, the IL-33 antagonist inhibits or reduces IL-33ox activity, thereby increasing protein expression levels of SCGB3A1. In some instances, the increased protein expression level is in the epithelium. In some instances, the increased expression is in the airway epithelium. In some instances, the increased protein expression level is in the upper airway epithelium. In some instances, the increased protein expression level is in ciliated pseudostratified columnar epithelium. In some instances, the increased protein expression level is in the lower airway epithelium. In some instances, the increased protein expression level is in the small airway epithelium. In some instances, the increased protein expression level is in cuboidal epithelium. In some instances, the increased protein expression level is in squamous epithelium.

Suitable samples and methods of measuring and determining an increase in protein expression levels of SCGB3A1 are disclosed elsewhere herein.

In some instances, reducing or preventing infections comprises increasing SCGB3A1 activity. In some instances, the IL-33 antagonist inhibits or reduces IL-33ox activity, thereby increasing SCGB3A1 activity. In some instances, the increased SCGB3A1 activity is in the epithelium. In some instances, the increased SCGB3A1 activity is in the airway epithelium. In some instances, the increased SCGB3A1 activity is in the upper airway epithelium. In some instances, the increased SCGB3A1 activity is in ciliated pseudostratified columnar epithelium. In some instances, the increased SCGB3A 1 activity is in the lower airway epithelium. In some instances, the increased SCGB3A 1 activity is in the small airway epithelium. In some instances, the increased SCGB3A 1 activity is in cuboidal epithelium. In some instances, the increased SCGB3A1 activity is in squamous epithelium.

In some instances, reducing or preventing infection comprises increasing mRNA expression levels of WFDC2. In some instances, the IL-33 antagonist inhibits or reduces IL-33ox activity, thereby increasing mRNA expression levels of WIFIC2. In some instances, the increased mRNA expression level is in the epithelium. In some instances, the increased expression is in the airway epithelium. In some instances, the increased expression is in the upper airway epithelium. In some instances, the increased expression is in ciliated pseudostratified columnar epithelium. In some instances, the increased expression is in the lower airway epithelium. In some instances, the increased expression is in the small airway epithelium. In some instances, the increased mRNA expression level is in cuboidal epithelium. In some instances, the increased mRNA expression level is in squamous epithelium. WFDC2 encodes WAP four-disulfide core domain 2 (WFDC2), which is an antiprotease with host cell defence functions that is expressed by club cells (Zuo et al Am J Respir Crit Care Med. 2018. 198:1375-1388). The examples show that treatment with an IL-33 antagonist increases expression of WFD)(2 from COPD epithelia. In some instances, the increased expression is in club 1 cells, club 2 cells, club 3 cells or club 4 cells. In some instances, the increased expression is in club 3 or club 4 cells.

Suitable samples and methods of measuring and determining an increase in mRNA expression levels of WFDC2 are disclosed elsewhere herein.

In some instances, reducing or preventing infection comprises increasing protein expression levels of WFDC2. In some instances, the IL-33 antagonist inhibits or reduces IL-33ox activity, thereby increasing protein expression levels of WFDC2. In some instances, the increased protein expression level is in the epithelium. In some instances, the increased expression is in the airway epithelium. In some instances, the increased protein expression level is in the upper airway epithelium. In some instances, the increased protein expression level is in ciliated pseudostratified columnar epithelium. In some instances, the increased protein expression level is in the lower airway epithelium. In some instances, the increased protein expression level is in the small airway epithelium. In some instances, the increased protein expression level is in cuboidal epithelium. In some instances, the increased protein expression level is in squamous epithelium.

Suitable samples and methods of measuring and determining an increase in protein expression levels of WFDC2 are disclosed elsewhere herein.

In some instances, reducing or preventing infections comprises increasing WFDC2 activity. In some instances, the IL-33 antagonist inhibits or reduces IL-33ox activity, thereby increasing WFDC2 activity. In some instances, the increased WFDC2 activity is in the epithelium. In some instances, the increased WFDC2 activity is in the airway epithelium. In some instances, the increased WFDC2 activity is in the upper airway epithelium. In some instances, the increased WFDC2 activity is in ciliated pseudostratified columnar epithelium. In some instances, the increased WFDC2 activity is in the lower airway epithelium. In some instances, the increased WFDC2 activity is in the small airway epithelium. In some instances, the increased WFDC2 activity is in cuboidal epithelium. In some instances, the increased WFDC2 activity is in squamous epithelium.

In some instances, reducing or preventing infection comprises increasing mRNA expression levels of MSMB. In some instances, the IL-33 antagonist inhibits or reduces IL-33ox activity, thereby increasing mRNA expression levels of MSMB. In some instances, the increased mRNA expression level is in the epithelium. In some instances, the increased expression is in the airway epithelium. In some instances, the increased expression is in the upper airway epithelium. In some instances, the increased expression is in ciliated pseudostratified columnar epithelium. In some instances, the increased expression is in the lower airway epithelium. In some instances, the increased expression is in the small airway epithelium. In some instances, the increased mRNA expression level is in cuboidal epithelium. In some instances, the increased mRNA expression level is in squamous epithelium. MSMB encodes beta-microseminoprotein, which is secreted by club cells (Zuo et al Am J Respir Crit Care Med, 2018, 198:1375-1388). The examples show that treatment with an IL-33 antagonist increases expression of beta-microseminoprotein from COPD epithelia. In some instances, the increased expression is in club 1 cells, club 2 cells, club 3 cells or club 4 cells. In some instances, the increased expression is in club 1, club 2 or club 3 cells.

Suitable samples and methods of measuring and determining an increase in mRNA expression levels of MSMB are disclosed elsewhere herein.

In some instances, reducing or preventing infection comprises increasing protein expression levels of beta-microseminoprotein. In some instances, the IL-33 antagonist inhibits or reduces IL-33ox activity, thereby increasing protein expression levels of beta-microseminoprotein. In some instances, the increased protein expression level is in the epithelium. In some instances, the increased expression is in the airway epithelium. In some instances, the increased protein expression level is in the upper airway epithelium. In some instances, the increased protein expression level is in ciliated pseudostratified columnar epithelium. In some instances, the increased protein expression level is in the lower airway epithelium. In some instances, the increased protein expression level is in the small airway epithelium. In some instances, the increased protein expression level is in cuboidal epithelium. In some instances, the increased protein expression level is in squamous epithelium.

Suitable samples and methods of measuring and determining an increase in protein expression levels of beta-microseminoprotein are disclosed elsewhere herein.

In some instances, reducing or preventing infections comprises increasing beta-microseminoprotein activity. In some instances, the IL-33 antagonist inhibits or reduces IL-33ox activity, thereby increasing beta-microseminoprotein activity, in some instances, the increased beta-microseminoprotein activity is in the epithelium. In some instances, the increased beta-microseminoprotein activity is in the airway epithelium. In some instances, the increased beta-microseminoprotein activity is in the upper airway epithelium. In some instances, the increased beta-microseminoprotein activity is in ciliated pseudostratified columnar epithelium. In some instances, the increased beta-microseminoprotein activity is in the lower airway epithelium. In some instances, the increased beta-microseminoprotein activity is in the small airway epithelium. In some instances, the increased beta-microseminoprotein activity is in cuboidal epithelium. In some instances, the increased beta-microseminoprotein activity is in squamous epithelium.

In some instances, reducing or preventing infection comprises increasing mRNA expression levels of LTF. In some instances, the IL-33 antagonist inhibits or reduces IL-33ox activity, thereby increasing mRNA expression levels of LTF. In some instances, the increased mRNA expression level is in the epithelium. In some instances, the increased expression is in the airway epithelium. In some instances, the increased expression is in the upper airway epithelium. In some instances, the increased expression is in ciliated pseudostratified columnar epithelium. In some instances, the increased expression is in the lower airway epithelium. In some instances, the increased expression is in the small airway epithelium. In some instances, the increased mRNA expression level is in cuboidal epithelium. In some instances, the increased mRNA expression level is in squamous epithelium. LTF encodes lactotransferrin, which is an anti-microbial protein with a variety of host cell defence functions. It is known to be expressed in the airway epithelium, for examples is submucosal secretory glands and the surface epithelium (Dubin et al Am J Physiol Lung Cell Mol Physiol 286: L750-L755, 2004). The examples show that treatment with an IL-33 antagonist increases expression of LTF in COPD epithelia. In some instances, the increased expression is in club 1 cells, club 2 cells, club 3 cells or club 4 cells. In some instances, the increased expression is in club 4 cells.

Suitable samples and methods of measuring and determining an increase in mRNA expression levels of LTF are disclosed elsewhere herein.

In some instances, reducing or preventing infection comprises increasing protein expression levels of lactotransferrin. In some instances, the IL-33 antagonist inhibits or reduces IL-33ox activity, thereby increasing protein expression levels of latetotransferrin. In some instances, the increased protein expression level is in the epithelium. In some instances, the increased expression is in the airway epithelium. In some instances, the increased protein expression level is in the upper airway epithelium. In some instances, the increased protein expression level is in ciliated pseudostratified columnar epithelium. In some instances, the increased protein expression level is in the lower airway epithelium. In some instances, the increased protein expression level is in the small airway epithelium. In some instances, the increased protein expression level is in cuboidal epithelium. In some instances, the increased protein expression level is in squamous epithelium.

Suitable samples and methods of measuring and determining an increase in protein expression levels of lactotransferrin are disclosed elsewhere herein.

In some instances, reducing or preventing infections comprises increasing lactotransferrin activity. In some instances, the IL-33 antagonist inhibits or reduces IL-33ox activity, thereby increasing latctotransferrin activity in the respiratory epithelium. In some instances, the increased lactotransferrin activity is in the epithelium. In some instances, the increased lactotransferrin activity is in the airway epithelium. In some instances, the increased lactotransferrin activity is in the upper airway epithelium. In some instances, the increased lactotransferrin activity is in ciliated pseudostratified columnar epithelium. In some instances, the increased lactotransferrin activity is in the lower airway epithelium. In some instances, the increased lactotransferrin activity is in the small airway epithelium. In some instances, the increased lactotransferrin activity is in cuboidal epithelium. In some instances, the increased lactotransferrin activity is in squamous epithelium.

In some instances, the IL-33 antagonist reduces or prevents infection by increasing club cell defence function in the airway epithelium. In some instances, increasing club cell defence function comprises increasing the mRNA expression levels of one or more club cell defence genes. In some instances, the one or more club cell defence genes are selected from the following list: SCGB1BA1, BP1FA1, SCGB3A1, WFDC2, MSMB, LTF, SLPI, C3, HLA-DRA, CXCL1, CD74, CXCL17, MDK, TGM2, HLA-DRB1, CXCL8, CXCL2, HLA-DRB5, CX3CL1 and HLA-DPA1. In some instances, the one or more club cell defence genes are selected from: SCGB1BA1, BP1FA1, SCGB3A1, WFDC2, MSMB and LTF. In some instances, the one or more club cell defence genes are SCGB1BA1 and BP1FA1. In some instances, increasing club cell defence function comprises increasing the protein expression levels of one or more proteins with club cell defence function selected from the following list: CCSP, SCGB3A1, WFDC2, Beta-microseminoprotein, lactotransferrin, SPLUNC1, SLPI, C3, HLA-DRA. CXCL1, CD74, CXCL17, MDK. TGM2, HLA-DRB1, CXCL8, CXCL2, HLA-DRB5, CX3CL1 and HLA-DPA1. In some instances, the one or more proteins with club cell defence function are selected from: CCSP. SCGB3A1, WFDC2, Beta-microseminoprotein, lactotransferrin and SPLUNC1. In some instances, the one or more proteins with club cell defence function are CCSP and SPLUNC1. In some instances, the club cell defence genes comprise SCGB1A1. LTF and/or BP1FA1.

In some instances, reducing or preventing infection comprises increasing mRNA expression levels of one or more of the markers selected from the following list: SLPI, C3, HLA-DRA, CXCL1, CD74, CXCL17, MDK, TGM2, HLA-DRB1, CXCL8, CXCL2, HLA-DRB5, CX3CL1 and HLA-DPA1.

In some instances, reducing or preventing infection comprises increasing protein expression levels of one or more of the following markers: secretory leukocyte protease inhibitor (SLPI), Complement C3, HLA-DR alpha chain, C—X—C motif chemokine ligand 1 (CXCL1), Cluster of Differentiation 74 (CD74), C—X—C motif chemokine 17 (CXCL17), midkine (MDK), Protein-glutamine gamma-glutamyltransferase 2 (TGM2), HLA class II histocompatibility antigen, DRB1 beta chain (HLA-DRB1), chemokine (C—X—C motif) ligand 8 (CXCL8), Chemokine (C—X—C motif) ligand 2 (CXCL2), HLA class II histocompatibility antigen, DRB5 beta chain (HLA-DRB5), chemokine (C-X3-C motif) ligand 1 (CX3CL1) and Major histocompatibility complex, class II, DP alpha 1 (HLA-DPA1).

In some instances, reducing or preventing infection comprises increasing activity of one or more of the following: SLPI, C3, HLA-DRA, CXCL1, CD74, CXCL17, MDK, TGM2, HLA-DRB1, CXCL8, CXCL2, HLA-DRB5, CX3CL1 and HLA-DPA1.

In some instances, reducing or preventing infection comprises increasing mRNA expression levels of one or more markers selected from the list consisting of: SLPI, C3, HLA-DRA, CXCL1, CD74, CXCL17, MDK. TGM2, HLA-DRB1, CXCL8, CXCL2, HLA-DRB5, CX3CL1 and HLA-DPA1.

In some instances, reducing or preventing infection comprises increasing protein expression levels of one or more markers selected from the list consisting of: SLPI, C3, HLA-DRA, CXCL1, CD74, CXCL17, MDK, TGM2, HLA-DRB1, CXCL8, CXCL2, HLA-DRB5, CX3CL1 and HLA-DPA1.

In some instances, reducing or preventing infection comprises increasing activity of one or more markers selected from the list consisting of: SLPI, C3, HLA-DRA, CXCL1, CD74, CXCL17, MDK, TGM2, HLA-DRB1, CXCL8, CXCL2, HLA-DRB5, CX3CL1 and HLA-DPA1.

In some instances, reducing or preventing infections reduces the annualised exacerbation rate in COPD. More than 50% of exacerbations are caused by respiratory tract viral infections. Therefore, improving club cell activity to thereby reduce the frequency of RTVI in subjects with COPD is likely to reduce the annualised exacerbation rate in the subject.

In some instances, reducing or preventing infections reduces the frequency of acute exacerbations of COPD (AECOPD).

In another aspect, the disclosure provides an IL-33 antagonist for use in a method pf treatment preventing or reducing respiratory tract infections in a subject with COPD. This is achieved by increasing club cell activity in the airway epithelium by inhibiting the activity of IL-33ox. The respiratory tract infection may be any of those described elsewhere herein. In particular instances, the respiratory tract infection may be a respiratory tract viral infection. The reduction in respiratory tract infections may be determined by monitoring the frequency of acute exacerbations of COPD (AECOPD) in a subject. If the number of AECOPD is statistically lower in a subject over a period of time following treatment, compared to the number of AECOPD over the same period of time prior to treatment, this indicates that the treatment has reduced respiratory tract infections in the subject. This is because more than 50% of AECOPD are caused by respiratory tract infections in COPD.

In some instances, the period of time is greater than 6 months. In some instance, the period of time is greater than 12 months. In some instances, the period of time is from 12 to 24 months. In some instances, the period of time is 18 months, 20 months, 22 or 24 months. In some instances, the period of time is 24 months.

In another aspect, the disclosure provides an IL-33 antagonist for use in a method of treatment reducing AECOPD in a subject with COPD, wherein the IL-33 antagonist attenuates or inhibits IL-33ox activity, thereby reducing respiratory tract infections in the subject.

In some instances, the attenuation or inhibition of IL-33ox activity increases club cell defence function, thereby reducing respiratory tract infections in the subject.

In some instances, the attenuation or inhibition of IL-33ox activity increases club cell defence function, thereby reducing respiratory tract infections in the subject

In some instances, the attenuation or inhibition of IL-33ox activity increases the mRNA expression levels of one or more of the markers described elsewhere herein, thereby reducing respiratory tract infections in the subject.

In some instances, the attenuation or inhibition of IL-33ox activity increases the protein expression levels of one or more of the markers described elsewhere herein, thereby reducing respiratory tract infections in the subject.

In some instances, the attenuation or inhibition of IL-33ox activity increases the activity of one or more proteins described herein with club cell defence functions, thereby reducing respiratory tract infections in the subject.

In some instances, the attenuation or inhibition of IL-33ox activity increases total club cell area in the epithelium of the subject, thereby reducing respiratory tract infections in the subject. In some instances, the epithelium is the airway epithelium. In some instances, the epithelium is the upper airway epithelium. In some instances, the epithelium is ciliated pseudostratified columnar epithelium. In some instances, the epithelium is lower airway epithelium. In some instances, the epithelium is small airway epithelium. In some instances, the epithelium is cuboidal epithelium. In some instances, epithelium is squamous epithelium.

### IL-33 Antagonists

The methods described herein comprise the use of an IL-33 antagonist.

In some instances, an IL-33 antagonist is a binding molecule. In some instances, the binding molecule specifically binds to IL33. Such a binding molecule is also referred to as an “IL-33 binding molecule” or an “anti-IL-33 binding molecule”. In some instances, the binding molecule specifically binds to IL-33 and inhibits or attenuates IL-33 activity.

In some instances, the IL-33 antagonist is an antibody or antigen binding fragment thereof. It is contemplated that antibodies or antigen binding fragments thereof that specifically bind to and inhibit components of the oxIL-33/RAGE/EGFR signaling axis may be useful in the methods disclosed herein.

In some instances, the binding molecule is antibody. In some instances, the antibody may be monoclonal (mAbs), recombinant, chimeric, humanized, such as complementarity-determining region (CDR)-grafted, human: antibody variants, including single chain, and/or bispecific, as well as antigen binding fragments, variants, or derivatives thereof. Antigen binding fragments include those portions of the antibody that bind to an epitope on the polypeptide of interest. Examples of such antigen binding fragments include Fab and F(ab′) fragments generated by enzymatic cleavage of full-length antibodies. Other antigen binding fragments include those generated by recombinant DNA techniques, such as the expression of recombinant plasmids containing nucleic acid sequences encoding antibody variable regions.

“Monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

A “chimeric” antibody refers to an antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In one instance, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeven et al., 1988, Science 239:1534-36), by substituting at least a portion of a rodent complementarity-determining region for the corresponding regions of a human antibody.

Also contemplated are human antibodies or antigen binding fragments thereof that bind to IL-33. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. Sec, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 54607381 and 546073A 1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In one instance, the antibodies are produced in mammalian host cells, such as CHO cells. Monoclonal (e.g., human) antibodies may be produced by the expression of recombinant DNA in host cells or by expression in hybridoma cells as described herein.

Antibodies and antigen binding fragments thereof useful in the present methods may comprise: (a) a heavy chain variable region comprising a HCDR1 having the sequence as set forth in SEQ ID NO: 1, a VHCDR2 having the sequence of SEQ ID NO: 2, a VHCDR3 having the sequence of SEQ ID NO: 3; and (b) a light chain variable region a VLCDR1 having the sequence of SEQ ID NO: 5, a VLCDR2 having the sequence of SEQ ID NO: 6, and a VLCDR3 having the sequence of SEQ ID NO: 7.

In some instances, the IL-33 antibody or antigen binding fragment thereof comprises a VH domain which comprises VHCDRs 1-3 of SEQ ID NO: 1, SEQ ID NO: 2 and SEQ ID NO: 3, respectively.

In some instances, the IL-33 antibody or antigen binding fragment thereof comprises a VH domain which comprises VHCDRs 1-3 consisting of SEQ ID NO: 1, SEQ ID NO: 2 and SEQ ID NO: 3, respectively.

In some instances, the anti-IL-33 antibody or antigen binding fragment thereof comprises HCDR1, HCDR2 and HCDR3 sequences of the VH domain having the sequence set forth in SEQ ID NO: 4.

In some instances, the IL-33 antibody or antigen binding fragment thereof comprises a variable heavy domain (VH) and a variable light domain (VL) having VL CDRs 1-3 having the sequences of SEQ ID NO: 5, 6 and 7, respectively, wherein one or more VLCDRs have 3 or fewer single amino acid substitutions, insertions and/or deletions.

In some instances, the IL-33 antibody or antigen binding fragment thereof comprises a VL domain which comprises VLCDRs 1-3 of SEQ ID NO: 5, SEQ ID NO: 6 and SEQ ID NO: 7, respectively.

In some instances, the IL-33 antibody or antigen binding fragment thereof comprises a VL domain which comprises VLCDRs 1-3 consisting of SEQ ID NO: 5, SEQ ID NO: 6 and SEQ ID NO: 7, respectively.

In some instances, the anti-IL-33 antibody or antigen binding fragment thereof comprises LCDR1, LCDR2 and LCDR3 sequences of the VL domain having the sequence set forth in SEQ ID NO: 8.

Also contemplated for use in the methods disclosed herein is an anti-IL-33 antibody or antigen binding fragment thereof comprising a heavy chain variable region (VH) domain at least 95%, 90%, or 85% identical to the sequence set forth in SEQ ID NO: 4. In some instances the anti-IL-33 antibody or antigen binding fragment thereof comprises a light chain variable region (VL) domain at least 95%, 90%, 85% identical to the sequence set forth in SEQ ID NO: 8. In some instances, the anti-IL-33 antibody or antigen binding fragment thereof comprises: (a) a heavy chain variable region (VH) at least 95%, 90%, or 85% identical to the sequence set forth in SEQ ID NO 4; and (b) a light chain variable region (VL) at least 95%, 90%, 85% identical to the sequence set forth in SEQ ID NO: 8. In some instances, the anti-IL-33 antibody is 33_640087_7B, as disclosed in WO2016/156440, which is incorporated herein by reference. 33_640087_7B, also referred to in the art as MEDI3506 or tozorakimab, is an anti-IL-33 antibody that binds to the reduced form of IL-33 (redIL-33) with high affinity. 33_640087_7B also inhibits the conversion of redIL-33 to the oxidised form (oxIL-33), which has been shown to induce signalling via RAGE and induce epithelial cell proliferation. A Ph3 clinical trial investigating the Efficacy and Safety of Tozorakimab (MEDI3506) in Symptomatic Chronic Obstructive Pulmonary Disease With a History of Exacerbations is currently ongoing (NCT05166889).

33_640087_7B is an exemplary anti-IL-33 antibody having: (a) a heavy chain variable region comprising a HCDR1 having the sequence as set forth in SEQ ID NO: 1, a VHCDR2 having the sequence of SEQ ID NO: 2, a VHCDR3 having the sequence of SEQ ID NO: 3; and (b) a light chain variable region a VLCDR1 having the sequence of SEQ ID NO: 5, a VLCDR2 having the sequence of SEQ ID NO: 6, and a VLCDR3 having the sequence of SEQ ID NO: 7.

33_640087_7B also comprises a VH domain having the amino acid sequence as set forth in SEQ ID NO: 4 and a VL domain having the amino acid sequence as set forth in SEQ ID NO: 8.

33_640087_7B is an IgG1 antibody, the sequence of the full length light chain and heavy chain of 33_640087_7B, including the IgGI chain, is set forth in SEQ ID NOs: 9 and 10, respectively.

Other exemplary IL-33 binding antagonists include anti-IL-33 antibodies or antigen binding fragments thereof, include ANB020, known as Etokimab (as described in WO2015/106080), itepekimab, 9675P (as described in US2014/0271658), A25-3H04 (as described in US2017/0283494), Ab43 (as described in WO2018/081075), IL33-158 (as described in US2018/0037644), 10C12.38.H6. 87Y.581 lgG4 (as described in WO2016/077381) or binding fragments thereof. Other exemplary anti-IL-33 antibodies or antigen binding fragments thereof include any of the other anti-IL-33 antibodies described in WO2016/156440, WO2015/106080, US2014/0271658, US2017/0283494, WO2018/081075, US2018/0037644 or WO2016/077381, all of which are incorporated herein by reference.

In some instances the anti-IL-33 antibody or antigen binding fragment thereof has similar, or the same pharmacokinetic (pK) characteristics as 33_670087_7B in humans.

In some instances the IL-33 binding molecule binds specifically to the reduced form of IL-33 (IL-33red), the oxidised form of IL-33 (IL-33ox) or both IL-33red and IL-33ox.

In some instances, the IL-33 binding molecule may attenuate or inhibit IL-33 activity by binding IL-33 in reduced or oxidised forms. In some instances, wherein the binding molecule inhibits or attenuates reduced IL-33 activity and oxidised IL-33 activity, this is achieved by binding to IL-33 in reduced form (i.e. by binding to reduced IL-33). In such instances, the binding molecule may bind to IL-33red and prevent its conversion to IL-33ox.

In some instances, the binding molecule may specifically bind to redIL-33 with a binding affinity (Kd) of less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M. 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In some instances, the binding affinity to redIL-33 is less than 5×10⁻¹⁴ M (i.e. 0.05 pM). In some instances, the binding affinity is as measured using Kinetic Exclusion Assays (KinExA) or BIACORE™. In some instances using KinExA, using protocols such as those described in WO2016/156440 (see e.g., Example 11), which is hereby incorporated by reference in its entirety. It has been found that binding molecules that bind to redIL-33 with this binding affinity bind tightly enough to prevent dissociation of the binding molecule/redIL-33 complex within biologically relevant timescales. Without wishing to be bound by theory, this binding strength is thought to prevent release of the antigen prior to degradation of the binding molecule/antigen complex in vivo, minimising any IL-33-dependent activity associated with IL-33 release from the binding complex.

In some instances, the binding molecule may specifically bind to redIL-33 with an on rate (k (on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. For example, a binding molecule of the disclosure may bind to redIL-33 or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹. In some instances, the k (on) rate is greater than or equal to 10⁷ M⁻¹ sec⁻¹. In some instances. the binding molecule may specifically bind to redIL-33 with an off rate (k(off)) of less than or equal to 5×10⁻¹ sec⁻¹, 10⁻¹ sec⁻¹, 5×10⁻² sec⁻¹, 10⁻² sec⁻¹, 5×10⁻³ sec⁻¹ or 10⁻³ sec⁻¹. For example, a binding molecule of the disclosure may be said to bind to redIL-33 or a fragment or variant thereof with an off rate (k(off)) less than or equal to 5×10⁻⁴ sec⁻¹, 10⁻⁴ sec⁻¹, 5×10⁻⁵ sec⁻¹, or 10⁵ sec⁻¹, 5×10⁻⁶ sec⁻¹, 10⁻⁶ sec⁻¹, 5×10⁻⁷ sec⁻¹ or 10⁻⁷ sec⁻¹. In some instances, the k (off) rate is less than or equal to 10⁻³ sec⁻¹. IL-33 is an alarmin cytokine released rapidly and in high concentrations in response to inflammatory stimuli. redIL-33 is converted to the oxidised approximately 5-45 mins after release into the extracellular environment (Cohen et al Nat Commun 6. 8327 (2015)). Without wishing to be bound by theory, binding to redIL-33 with these k (on) and/or k (off) rates may minimize exposure to redIL-33 prior to conversion of the reduced from to oxIL-33. Moreover, the k (off) rate may prevent IL-33 release from the binding molecule/antigen complex prior to degradation of the complex in vivo. These binding kinetics may also act to prevent conversion of redIL-33 to oxIL-33, and thus prevent pathological signaling of the oxidised form of IL-33 via RAGE/EGFR (as described in WO2021/089563, which is incorporated herein by reference).

In some instances, the IL-33 antibody or antigen binding fragment thereof may competitively inhibit binding of IL-33 to 33_640087-7B (as described in WO2016/156440). WO2016/156440 discloses that 33_640087-7B binds to redIL-33 with particularly high affinity and attenuates both ST-2 and RAGE-dependent IL-33 signaling. An antibody or antigen binding fragment thereof is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays. Dissociation-Enhanced Lanthanide Fluorescent Immunoassays (DELFIA®. Perkin Elmer), and radioligand binding assays. For example, the skilled person could determine whether an antibody or antigen binding fragment thereof competes for binding to IL-33 by using an in vitro competitive binding assay, such as the HTRF assay described in WO2016/156440, paragraphs 881-886, which is incorporated herein by reference. For example, the skilled person could label 33_640087-7B with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding antibody molecules, the skilled person could first mix various concentrations of a test binding molecule with a fixed concentration of the labelled 33_640087-7B antibody. A reduction in the FRET signal when the mixture is incubated with labelled IL-33 in comparison with a labelled antibody-only positive control would indicate competitive binding to IL-33. An antibody or antigen binding fragment thereof may be said to competitively inhibit binding of the reference antibody to a given epitope by at least 90%, at least 80%, at least 70%, at least 60%, or at least 50%.

In various instances, the anti-IL-33 antibody or antigen binding fragment thereof selected from human antibody, a humanized antibody, a chimeric antibody, a monoclonal antibody, a recombinant antibody, an antigen-binding antibody fragment, a single chain antibody, a monomeric antibody, a diabody, a triabody, tetrabody, a Fab fragment, an lgG1 antibody, an lgG2 antibody, an lgG3 antibody, and an lgG4 antibody. In some instances, the anti-IL-33 antibody or antigen binding fragment is selected from the group consisting of a diabody, a triabody, a tetrabody, a Fab fragment, single domain antibody, scFv, wherein the dose is adjusted such that the binding sites to be equimolar to those dosed by bivalent antibodies.

In some instances, the anti-IL-33 antibody or antigen binding fragment thereof binds to IL-33 comprising an amino acid sequence of SEQ ID NO:11. In various instances, the anti-IL-33 antibody or antigen binding fragment thereof may be capable of binding to a mature form of the full-length IL-33 protein comprising an amino acid sequence of SEQ ID NO:11. In various instances, the anti-IL-33 antibody or antigen binding fragment thereof may be capable of binding to an IL-33 protein fragment comprising amino acids 72-270, 79-270, 95-270, 99-270, 107-270, 109-270, 111-270, or 112-270 of SEQ ID NO:11.

In various instances, the anti-IL-33 antibody or antigen binding fragment thereof may be capable of binding to the reduced (red-IL-33) and/or the oxidised (ox-IL-33) form of IL-33. In some instances, the anti-IL-33 antibody or antigen binding fragment thereof may be capable of preferentially binding to the reduced (red-IL-33) and/or the oxidised (ox-IL-33) form of IL-33.

In various instances, the anti-IL-33 antibody or antigen binding fragment thereof may be an inhibitory antibody, capable of inhibiting IL-33 or a fragment thereof as defined herein. In various instances, an inhibitory antibody may be capable of inhibiting the association of IL-33 or a fragment thereof with an IL-33 receptor.

In some instances, the anti-IL-33 antibody comprises a light chain sequence as set forth in SEQ ID NO: 9 and a heavy chain sequence as set forth in SEQ ID NO: 10.

In some instances, the anti-IL-33 antibody comprises a light chain having the sequence as set forth in SEQ ID NO:9 and a heavy chain having the sequence as set forth in SEQ ID NO: 10.

In some instances, the anti-IL-33 antibody comprises a light chain consisting of the sequence as set forth in SEQ ID NO:9 and a heavy chain consisting of the sequence as set forth in SEQ ID NO: 10.

In some instances, the binding molecule inhibits IL-33ox activity. In some instances, the binding molecules inhibits bindings of IL-33ox to the RAGE/EGFR complex.

### Compositions and Administration

The IL-33 antagonists in the medical uses and methods described herein may be administered to a patient in the form of a pharmaceutical composition.

Suitably, any references herein to ‘a/the IL-33 antagonist’ may also refer to a pharmaceutical composition comprising an/the IL-33 antagonist. Suitably the pharmaceutical composition may comprise one or more IL-33 antagonists.

Suitably the IL-33 antagonist may be administered in a pharmaceutically effective amount for the in vivo treatments described herein.

Suitably, the IL-33 antagonist or a pharmaceutical composition thereof may be administered to a human or other animal in accordance with the aforementioned methods of treatment/medical uses in an amount sufficient to produce a therapeutic effect.

Suitably, the IL-33 antagonist or a pharmaceutical composition thereof can be administered to such human or other animal in a conventional dosage form prepared by combining the IL-33 antagonist with a conventional pharmaceutically acceptable carrier or diluent according to known techniques.

It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of IL-33 antagonists may prove to be particularly effective.

The amount of IL-33 antagonist that may be combined with the carrier materials to produce a single dosage form will vary depending upon the subject treated and the particular mode of administration. Suitably, the pharmaceutical composition may be administered as a single dose, multiple doses or over an established period of time in an infusion. Suitably, dosage regimens also may be adjusted to provide the optimum desired response (e.g., a therapeutic or prophylactic response).

Suitably, the IL-33 antagonist will be formulated so as to facilitate administration and promote stability of the IL-33 antagonist.

Suitably, pharmaceutical compositions are formulated to comprise a pharmaceutically acceptable, non-toxic, sterile carrier such as physiological saline, non-toxic buffers, preservatives and the like.

Suitably the pharmaceutical composition may comprise pharmaceutically acceptable carriers, sterile aqueous or non-aqueous solutions, suspensions, and/or emulsions.

Suitably, pharmaceutical compositions for injectable use may include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will be preserved against the contaminating action of microorganisms, such as bacteria and fungi.

Suitable formulations for use in the therapeutic methods disclosed herein are described in Remington's Pharmaceutical Sciences (Mack Publishing Co.) 16th ed. (1980).

Suitably, prevention of the action of microorganisms can be achieved by various antibacterial and antifungal agents. In many cases, it will be suitable to include isotonic agents, in the pharmaceutical composition. Prolonged absorption of the injectable compositions can be brought about by including in the composition an agent which delays absorption.

Suitably, sterile injectable solutions can be prepared by incorporating an IL-33 antagonist in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation may be vacuum drying and freeze-drying, which yields a powder of an active ingredient plus any additional desired ingredient from a previously sterile-filtered solution thereof.

Methods of administering the IL-33 antagonist or a pharmaceutical composition thereof to a subject in need thereof may be readily determined by those skilled in the art.

Suitably, the route of administration of the IL-33 antagonist or pharmaceutical composition thereof may be, for example, oral, parenteral, by inhalation or topical. Suitably, the term parenteral as used herein includes, e.g., intravenous, intraarterial, intraperitoneal, intramuscular, subcutaneous, rectal, or vaginal administration.

Suitably, the IL-33 antagonist or pharmaceutical composition thereof may be orally administered in an acceptable dosage form including, e.g., capsules, tablets, aqueous suspensions or solutions. Suitably, parenteral formulations may be a single bolus dose, an infusion or a loading bolus dose followed with a maintenance dose. These compositions may be administered at specific fixed or variable intervals, e.g., once a day, or on an “as needed” basis.

Suitably, the components as recited hereinabove for preparing a pharmaceutical composition described herein may be packaged and sold in the form of a kit. Such a kit will suitably have labels or package inserts indicating that the associated pharmaceutical compositions are useful for treating a subject suffering from, or predisposed to a disease or disorder.

### EXAMPLES

The airway epithelium plays a central role in the initiation and development of chronic airway diseases (Carlier et al Front. Physiol. 12, 691227 (2021)). Constant exposure to pathogens and noxious stimuli alters the structure and composition of airway epithelia and may lead to irreversible changes, such as those occurring in chronic airway disease (Carlier et al Front. Physiol. 12, 691227 (2021): Hogg et al Annu. Rev. Pathol. 4, 435-459 (2009)). Genetic analyses suggest that IL-33 drives the pathology of chronic airway diseases such as asthma and COPD: a rare loss-of-function mutation in IL-33 reduces the risk of asthma and COPD, whereas gain-of-function mutations are associated with an increased risk of COPD (Rabe et al Lancet Respir. Med. 9, 1288-1298 (2021): Smith et al PLOS Genet. 13, e1006659 (2017)). IL-33 binds to cell surface IL-1 receptor-like 1 (IL1RL1, also known as ST2), activating NF-κB inflammatory signalling pathways and leading to chronic airway inflammation (Liew et al Nat. Rev. Immunol. 16, 676-689 (2016).

The impact of IL-33 on immune cells is well established. However, more recent evidence has emerged implicating IL-33 having a direct effect on epithelial cells. In vitro epithelial damage assays and validated IL-33 pathway blocking reagents have shown that the oxidised form of IL-33 (oxIL-33 or IL-33ox) signals through a newly described RAGE/EGFR signalling pathway, which transforms the functional dynamics of airway epithelium (as disclosed in WO 2021/089563, which is incorporated herein by reference).

In the present study, transcriptional dynamics in the airway epithelium were interrogated to further explain the role of RAGE/EGFR complex in health and disease.

### IL-33^{ox} Redirects Epithelial Cell Fate

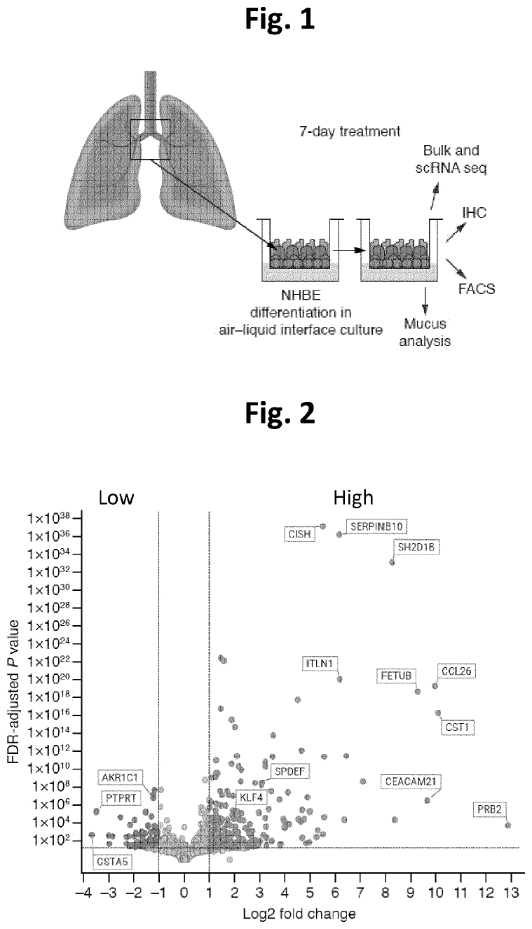

To investigate IL-33°N-driven transcriptional changes in lung epithelia, normal human bronchial epithelial (NHBE) cells from healthy donors were differentiated into air-liquid interface (ALI) cultures, modelling human epithelial physiology (Zscheppang et al Biotechnol. J. 13, 1700341 (2018))(FIG. 1).

IL-33^{ox} treatment induced a plethora of transcriptional changes, in contrast to no treatment (FIG. 2).

IL-33^{ox} decreased expression of genes associated with epithelial cell differentiation and increased expression of genes associated with negative regulation of wound closure (data not shown). Genes associated with mitochondrial organization, ATP metabolism, endoplasmic reticulum/Golgi vesicle transport and cellular stress markers were also upregulated (data not shown).

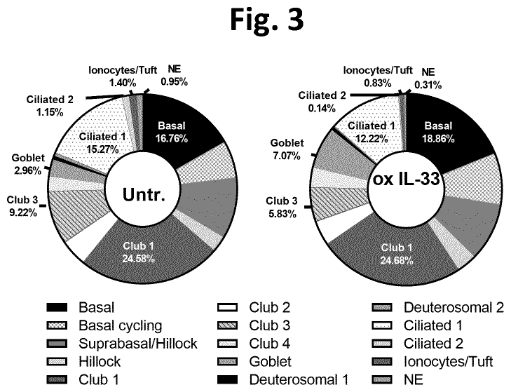

Next, we investigated the transcriptional changes induced by IL-33^{ox} in ALI cultures derived from healthy donors at the single-cell level (Hewitt et al Nat. Rev. Immunol. 21, 347-362 (2021)). We identified 15 cell states in healthy untreated ALI cultures that were representative of the major airway epithelial cell types and consistent with the complex cell heterogeneity observed in vivo (Jackson et al Cell Rep. 32, 107872 (2020); Ruiz Garcia et al Development 146, dev177428 (2019))(Table 1). The proportion of secretory and basal cells increased in healthy ALI cultures after treatment with IL-33^{ox}, whereas that of ciliated and rare cell types decreased (FIG. 3). Within the secretory cell population, the proportion of mucus-producing cells increased after IL-33^{ox} treatment, whereas that of mature club cells (club 3, involved in epithelial defence and expressing high levels of SCGB1A1 and SCGB3A1 (Zuo et al Crit. Care Med. 198, 1375-1388 (2018)) decreased.

TABLE 1
Fifteen cell states identified in healthy ALI cultures
representing cell heterogeneity observed in vivo

| Cell types | Cell states | Gene marker |
|---|---|---|
| Basal | Basal | KRT5+/KRT15+/TP63+/BCAM+/COL17A1+ |
| cells | Basal cycling | KRT5+/MKI67+/TOP2A+/CENPF+ |
| | Suprabasal/Hillock | KRT5+/KRT13+/S100A2+/TXN+/SERPINB4+ |
| | Hillock | KRT13+/KRT4+/SPRR1B+ |
| Secretory | Club 1 (immature) | SCGB3A1+(low)/AQP5+/CLDN4+/HES1+/HES4+ |
| cells | Club 2 (immature) | SCGB3A1+(low)/HES4+/PIGR+ |
| | Club 3 (mature) | SCGB1A1+/SCGB3A1+/BPIFA1+/BPIFB1+/MSMB+/VMO1+ |
| | Club 4 (immune | SAA1+/SAA2+/CEACAM6+/LCN2+/C3+ |
| | response) | |
| | Goblet | MUC5AC+/TFF3+/SPDEF+/FCGBP+/XBP1+ |
| Ciliated | Deuterosomal 1 | CDC20B+/E2F7+/PLK4+/HES6+/DEUP1+/CCNO+ |
| cells | (intermediate ciliated | |
| | cell 1) | |
| | Deuterosomal 2 | DEUP1+/CCNO+/CCNA1+/MUC12+/HIST1H1C+/HES6+/SMIM6+ |
| | (intermediate ciliated | |
| | cell 2) | |
| | Ciliated 1 | PIFO+/DNAH5+/RSPH1+/C20orf851+/TUBA1A+/TMEM190+ |
| | Ciliated 2 | PIFO+/DNAAF13+/BEST4+/DNAH124+/LRRIQ1+ |
| Rare cell | Ionocytes/tuft | FOXI1+/DCLK1+/ASCL3+/HES6+/CFTR+ |
| types | cells | |
| | Neuroendocrine | ASCL1+/CALCA+/CHGA+/HOXB2+ |
| | (pulmonary | |
| | neuroendocrine | |
| | cells) | |

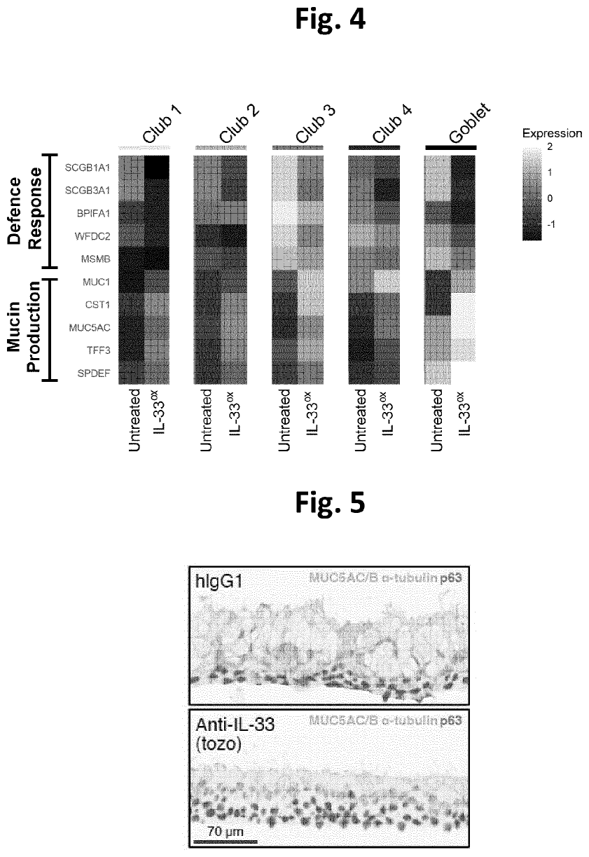

Differential gene expression analysis showed that club-associated genes involved in epithelial defence functions (e.g. SCGB1A1, SCGB3A1, BP1FA1, WFDC2 and MSMB (Zuo et al Crit. Care Med. 198, 1375-1388 (2018); Akram et al Mucosal Immunol. 11, 71-81 (2018); Goldfarbmuren et al Nat. Commun. 11, 2485 (2020)) were downregulated in all secretory cells (FIG. 4). Notably, the most highly differentially expressed genes in the single-cell data were consistent with data from bulk RNA sequencing (data not shown). Collectively, these data suggest that IL-33°N drives epithelial remodelling at the expense of club and ciliated cell states, likely reducing inherent epithelial defence functions against infections. Such a phenotype may lead to increased risk of respiratory tract infections in subjects with COPD, which is a major cause of acute exacerbation events in COPD.

### Blocking IL-33 Reverses COPD Key Features

Epithelial changes induced by chronic exposure to exogenous IL-33°N were reminiscent of those observed in the airway epithelium of patients with COPD (Kesimer et al N. Engl. J. Med. 377, 911-922 (2017); Ha et al Pharmacology 97, 84-100 (2016); Boucher N. Engl. J. Med. 380, 1941-1953 (2019); Kim et al Am. J. Respir. Crit. Care Med. 187, 228-237 (2013)).

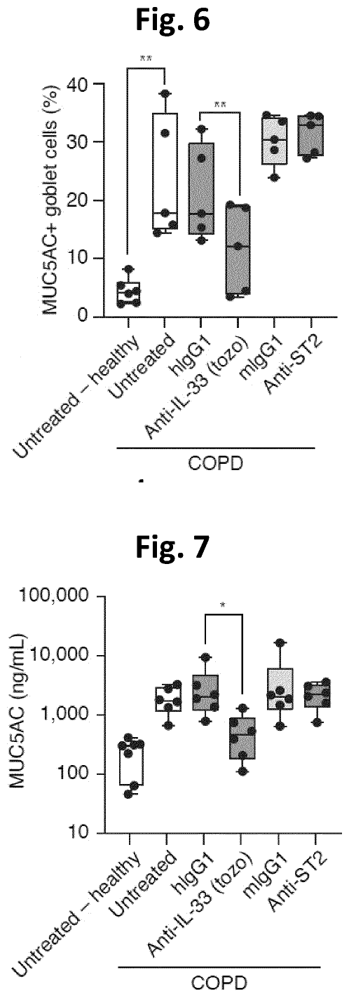

Inhibition of IL-33 with tozorakimab in COPD ALI cultures reduced the proportion of mucus-secreting cells and amount of MUC5AC released (FIG. 5-7). These effects were not observed after selectively blocking ST2 (data not shown), suggesting that IL-33ON may be the driver of this phenotype.

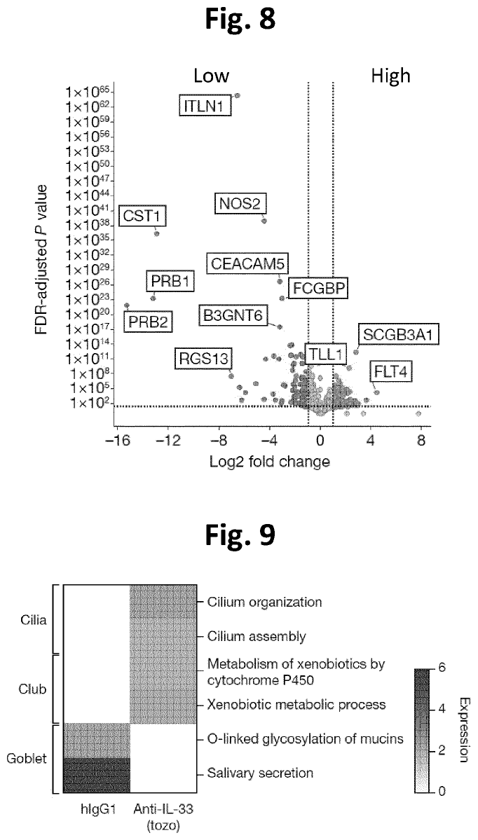

Furthermore, inhibition of IL-33^{ox} signalling induced substantial transcriptomic changes, restoring genes associated with cilia and club cells, thereby reversing the COPD phenotype of ALI cultures (FIGS. 8 and 9). We observed downregulation of genes known to be associated with goblet cell differentiation and carbohydrate biosynthesis, and upregulation of genes associated with detoxification functions, club cells and cilia organization and assembly (FIG. 9).

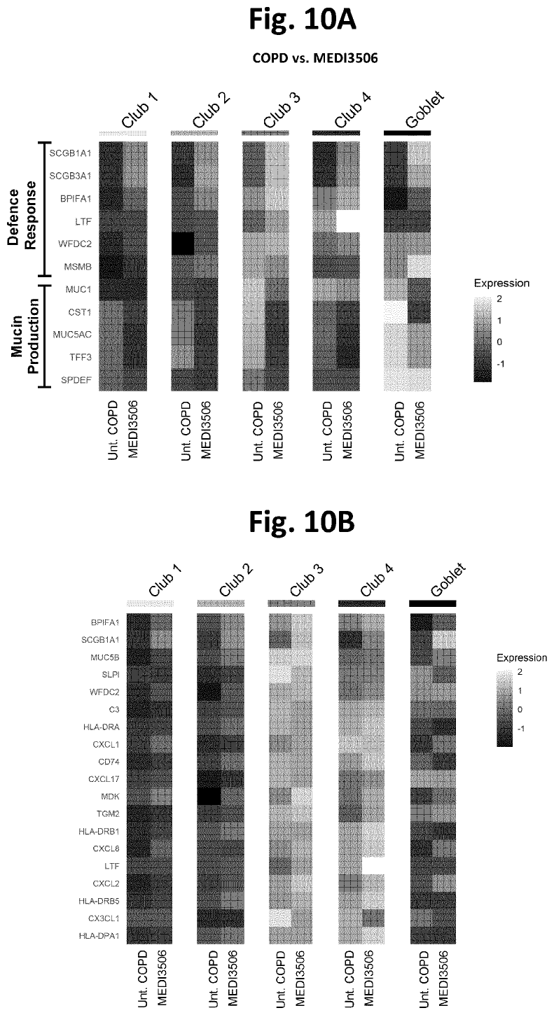

Using single-cell transcriptomics, we investigated the effects of blocking endogenous IL-33 signalling in COPD ALI cultures (FIG. 10,). Although major changes in proportions of different cell states were not observed, inhibition of IL-33 signalling upregulated genes associated with epithelial host defence functions and club cell markers (e.g. SCGB1A1 and SCGB3AI)(Mootz et al Allergy https://doi.org/10.1111/all.15033, https://doi.org/10.1111/all. 15033 (2021))(FIG. 10).

### DISCUSSION

This study reveals a previously unknown role of IL-33^{ox} that, through a newly described RAGE/EGFR signalling pathway, transforms the transcriptional and functional dynamics of airway epithelium. We hypothesize that IL-33^{ox} acts to protect the lung during acute injury or infection, but that excessive exposure during chronic damage disrupts the normal repair processes, leading to epithelial dysfunction, mucus hypersecretion and pathogenesis. Recent clinical data indicate a clinical benefit of inhibiting IL-33 in patients with COPD (Rabe et al. Based on our findings, we speculate that therapies designed to inhibit signalling of both IL-33^{ox} and IL-33^{red} will have greater clinical impact than those targeting IL-33^{red}/ST2-induced inflammation alone. Furthermore, our results suggest that tozorakimab can revert pathogenic traits in COPD epithelia and restore club cell defence mechanisms, thereby reducing infections that lead to COPD exacerbations, reducing patient hospitalisations and increasing quality of life.

### Materials and Methods

### Cell Culture

### NHBE Cells

NHBE cells (Lonza, CC-2540) were cultured in complete BEGM (Lonza, CC-3171) with the supplement kit (Lonza, CC-4175) according to the manufacturer's protocol.

### ALI Cultures

Transwells containing 12 mm or 6.5 mm 0.4-μm polyester membrane inserts (Corning, CLS3460 and CLS3470) were coated with CellAdhere Type I Collagen (Stemcell, 07001) diluted once in distilled H₂O and incubated at 37° C. for 1-16 h, then washed with PBS.

Lung epithelial cells from healthy controls (bronchial [Lonza, CC-2540] or small airway [Epithelix, EP61SA]) or patients with COPD (bronchial [Lonza, 195275] or small airway [Epithelix, EP66SA]) were grown in four T-175 flasks in Epix Medium (Propagenix, 276-201) for bronchial cells or small airway epithelial cell growth medium (PromoCell, C-21070) for small airway epithelial cells. Once confluent, cells were frozen down at 1×10⁶ cells/vial at passage 2. Cells at passage 2 were plated in two T-75 flasks, grown until 80% confluent, and washed and detached using 6 ml trypsin (Lonza, CC-5034). The cell suspension was centrifuged at 1,200 RPM for 5 min and cells were resuspended in PneumaCult ALI medium (Stemcell, 05001) for bronchial cells or PneumaCult ALI-S medium (Stemcell, 05050) for small airway cells at 8×10⁵ cells/ml; 0.5 ml and 0.25 ml were dispensed onto each 12 mm and 6.5 mm insert, respectively, and 1 ml or 0.5 ml of ALI medium were added into the space below the respective inserts. Cells were maintained in ALI medium until tight junctions were formed. Medium was then removed from the apical side and cells were differentiated for 3 weeks, with medium changed on the basal side every 2-3 days.

Fully differentiated healthy cultures were left untreated or treated with IL-33^{ox} (30 ng/ml), untagged IL-33^{C>S} (30 ng/ml) or EGF (30 ng/ml). Differentiated COPD cultures were left untreated or treated with 1 μg/ml tozorakimab. 1 μg/ml NIP228 (IgGI isotype control), 10 μg/ml mNIP228, 10 μg/ml anti-ST2, 1 μg/ml anti-RAGE (4F4), 1 μg/ml anti-EGFR (Millipore, clone LA1) or RAGE-FC (R&D Systems). For both healthy and COPD ALI cultures, treatment was supplied in the medium on the basal side for 7 days. Medium was changed every 2-3 days. Antibodies used in this study are listed in

Supplementary Table 5. Tozorakimab directly inhibits IL-33^{red}/ST2L signalling by preventing IL-33^{red} from interacting with ST2L, and it indirectly inhibits IL-33^{ox}-RAGE/EGFR signalling by preventing the formation of oxidized IL-33 (E.S.C. manuscript in preparation).

### Recombinant Protein Production

### Cloning and Expression of IL-33

cDNA molecules encoding the mature component of wild-type (WT) human IL-33 (aa 112-270), UniProt accession number 095760 (IL-33red), and a variant with all four cysteine residues mutated to scrine (IL-330^{C>S}) that is resistant to oxidation were synthesized by primer extension PCR and cloned into pJexpress 411 (DNA 2.0). WT IL-33 was considered to be in its reduced form (IL-33^{red}) in 2×DPBS storage buffer before addition to culture medium. Sequences of both were modified to contain a 10×His, Avitag and Factor Xa protease cleavage site (MHHHHHHHHHHAAGLNDIFEAQKIEWHEAAIEGR (SEQ ID NO:12)) at the N-terminus. IL-33^{red} (N-terminal tagged His10/Avitag: WT, SEQ ID NO:13) and IL-33^{C>S} (N-terminal tagged His 10/Avitag: WT, SEQ ID NO: 14) were generated by transforming Escherichia coli BL21 (DE3) cells, which were cultured in autoinduction medium (Overnight Express Autoinduction System 1, Merck Millipore, 71300-4) at 37° C. for 18 h, harvested by centrifugation and stored at −20° C. Cells were resuspended in 2×DPBS containing complete EDTA-free protease inhibitor cocktail tablets (Roche, 11697498001) and 50 U/ml Benzonase nuclease (Merck Millipore, 70746-3), and lysed by sonication. Cell lysate was centrifuged at 50,000×g for 30 min at 4° C. IL-33 proteins were purified from the supernatant by immobilized metal affinity chromatography and further purified by size-exclusion chromatography (SEC) using a HiLoad 26/600 Superdex 75 pg column (GE Healthcare, 28989334). Peak fractions were analysed by SDS-PAGE. Fractions containing pure IL-33 were pooled and their concentrations determined by measuring absorbance at 280 nm. Final samples were analysed by SDS-PAGE. To generate untagged IL-33^{red} or IL-330^{C>S}, N-terminal tagged His 10/Avitag IL-33 was incubated with 10 units of Factor Xa (GE Healthcare, 27084901) per mg of protein in 2×DPBS at room temperature for 1 h. Untagged IL-33 was purified using SEC on a HiLoad 16/600 Superdex 75 pg column (GE Healthcare, 28989333) with a flow rate of 1 ml/min.

### Generation and Purification of IL-33^{ox}

IL-33^{red} was oxidized by dilution to a final concentration of 0.5 mg/ml in 60% IMDM (with no phenol red) and 40% DPBS. Tags were cleaved from IL-33^{ox} by incubation with Factor Xa (NEB, P8010L) at a final concentration of 1 μg/50 μg of IL-33^{ox} for 120 min at 22° C. To deplete the sample of any remaining IL-33^{red}, soluble human ST2 fused to human IgG1 Fc-His6 was incubated with the sample for 30 min at 22° C. The sample was concentrated and loaded on a HiLoad 26/600 Superdex 75 pg column (GE Healthcare, 28989334) at a flow rate of 2 ml/min. Each fraction containing pure IL-33^{ox} was tested for its ability to activate EGFR (homogeneous time-resolved fluorescence [HTRF] assay in A549 cells and NHBE cells). Active fractions were pooled and concentrated, and the final concentration of the sample was determined using UV absorbance spectroscopy at 280 nm. Final product quality was assessed by SDS-PAGE, high-performance SEC and reverse-phase HPLC.

qPCR

Following 7 days of treatment, 4-week-old healthy or COPD ALI cultures on 6.5 mm inserts were lysed for RNA analysis. Each ALI apical surface was incubated for 30 min at 37° C. with 200 μl PBS. Direct-zol RNA Miniprep kits (Zymo Research, R2050) were used for RNA extraction. For submerged cultures (A549 cells, HUVECs and NHBE cells) the RNeasy Mini Kit (Qiagen, 74104) was used. cDNA was synthesized using the High-Capacity RNA-to-cDNA Kit (Thermo, 4388950).

For RT-qPCR, 4 μl cDNA, 5 μl TaqMan Fast Advanced Master Mix (Thermo, 4444557), 0.5 μl MUC5AC FAM probe (Thermo, Hs01365616_ml) or MUC2 (Thermo, Hs00894041_g1) or CST1 (Thermo, Hs00606961_ml) or ST2 long (Thermo, Hs00249389_ml) or ST2 short (Thermo, Hs01073297_ml), and 0.5 μl GAPDH VIC probe (Thermo, Hs02786624_g1) were added to a MicroAmp EnduraPlate (Thermo, 4483273). Plates were sealed and briefly centrifuged before analysis using a QuantStudio 7 Flex Real-Time PCR system (Thermo). ΔΔCT was calculated by normalizing data to an untreated control.

### Bulk RNA Sequencing

RNA extracted from ALI cultures was processed externally by Source BioScience (Cambridge, UK). The library was prepared using the Illumina mRNA stranded kit. Sequencing was performed on an Illumina NovaSeq 6000 System to generate 30M 150-base-pair paired-end reads. RNA libraries were prepared in accordance with the NEBNext Ultra II Directional RNA Sample Preparation Protocol for Illumina Paired-End Multiplexed Sequencing.

Sequenced libraries were checked for quality using MultiQC⁴⁸ based on STAR⁴⁹ alignment against the GRCh38 ensembl (v100) human genome. Adapter trimming was performed using NGmerge⁵⁰), and Salmon⁵¹ was used for gene expression quantification using GRCh38 ensembl (v100) as a reference. The bioinformatics workflow was organized using Nextflow⁵² and Bioconda software management tools⁵³. Differential expression analysis was performed in R using the DESeq2⁵⁴ package with “apeglm”⁵⁵ fold change shrinkage. The Benjamini-Hochberg method was used for multiple correction of P values⁵⁶. Volcano plots showing the fold change and q-value were created using Spotfire (TIBCO) data analysis software. Gene Set Variation Analysis (GSVA) 57 was used to calculate samplewise gene set enrichment scores for the generated signatures in public COPD patient gene expression data sets GSE37147⁴⁴, GSE11784⁴⁶ and GSE47460⁴⁵. Calculations were performed using the GSVA package in R. Patient groups were compared according to disease and smoking status for gene sets GSE37147 and GSE11784, and according to COPD severity by GOLD stage for GSE47460. Significance was calculated using one-way ANOVA, followed by post hoc pairwise comparisons with Tukey's honest significant difference test conducted in Prism 9 (GraphPad).

### Single-Cell RNA Sequencing

Single-cell suspensions of ALI cultures were generated as described in the FACS analysis section, washed twice with PBS/0.04% BSA and resuspended at 900 cells/μl. Cells were mixed with reagents from Chromium Next GEM Single Cell 3′ GEM Kit v3.1 (10× Genomics, 1000123), as per the manufacturer's instructions. Both the sample mix and capture beads from Chromium Next GEM Single Cell 3′ Gel Bead Kit v3.1 (10× Genomics, 1000122) were loaded onto the microfluidic chip-Chromium Next GEM Chip G (10× Genomics. 2000177)-aiming to capture 8000 cells per sample. The chip was run on a Chromium Single Cell Controller (10× Genomics, GCG-SR-1) for single-cell partitioning and barcoding, and cDNA was prepared from the barcoded cells using Chromium Next GEM Single Cell 3′ GEM Kit v3.1 (10× Genomics, 1000123). Data were aligned to GRCh38-3.0.0 human reference genome using CellRanger v3.0.1 (10× Genomics). Normalization and downstream analyses were performed using the Seurat v3.2.358 package in R v3.6.3. Raw counts were normalized and scaled using the Seurat functions NormalizeData and ScaleData (default parameters). Uniform Manifold Approximation and Projection (UMAP) dimensionality reduction was obtained by applying Seurat's RunUMAP function considering the top 2.000 most variable genes, and the 25 first principal components (PCAs) were obtained using Seurat's RunPCA function. For the healthy donor (n=1), cell clustering was performed by applying Seurat's functions FindNeighbors and FindClusters. A resolution of 0.5 was applied, as this refined a cluster containing goblet cell identity. Clusters that showed high proximity in the phylogenetic tree obtained using BuildClusterTree were merged. Cell cluster markers were obtained with Seurat's FindAllMarkers function (non-parametric Wilcoxon rank sum test) and used for cluster annotation. Cell types were annotated manually based on the highly expressed genes for each cluster and known epithelial airway cell markers (Supplementary FIG. 1 and Supplementary Table 3)^{17,20,59-61}. The healthy donor was used as the reference to project cell annotations on to the COPD donors (n=3) with Seurat's TransferData function. All differential gene expression analyses between distinct treatments were performed using non-parametric Wilcoxon rank sum tests using the Seurat functions FindMarkers and FindAllMarkers. Genes considered to be differentially expressed showed log-fold change cut-offs of 0.5 and adjusted P values for multiple testing below 0.001 (Bonferroni correction).

### Statistical Analysis

Statistical analyses were conducted using R (v.4.0.2) or Prism 9 (GraphPad), which were also used to generate the plots. Data are presented as mean with standard error of the mean and a one-way ANOVA followed by a Tukey's test for comparisons between more than two groups, unless indicated otherwise. A significance threshold of 0.05 was used for P values. Box plots were generated using the following parameters: horizontal black lines with each box present median values: boxes extend from 25th to 75th percentiles of values: whiskers extend to a maximum of 1.5× the interquartile range (75th percentile-25th percentile) beyond the boxes: lowest dots are minimum values and highest dots are maximum values for each box. All experiments are represented by several biological replicates or independent experiments, unless otherwise mentioned. The number of replicates per experiment is indicated in the legends. The quantitative Venn diagram of mass spectrometry data was created using the Bioinformatics & Evolutionary Genomics web tool⁶². All western blots, co-immunoprecipitation experiments, FACS analyses, ELISAs and RT-qPCRs were independently replicated at least twice with similar results. No statistical methods were used to predetermine sample size.

### ADDITIONAL REFERENCES

17 Jackson, N. D. et al. Single-cell and population transcriptomics reveal pan-epithelial remodeling in type 2-high asthma. Cell Rep. 32, 107872 (2020).
20 Ruiz Garcia, S. et al. Novel dynamics of human mucociliary differentiation revealed by single-cell RNA sequencing of nasal epithelial cultures. Development 146i, dev 177428 (2019).
44 Steiling. K. et al. A dynamic bronchial airway gene expression signature of chronic obstructive pulmonary disease and lung function impairment. Am. J. Respir. Crit. Care Med. 187, 933-942 (2013).
45 Tan, J. et al. Expression of RXFP1 is decreased in idiopathic pulmonary fibrosis. Implications for relaxin-based therapies. Am. J. Respir. Crit. Care Med. 194, 1392-1402 (2016).
46 Tilley, A. E. et al. Biologic phenotyping of the human small airway epithelial response to cigarette smoking. PLOS One 6, e22798 (2011).
48 Ewels, P., Magnusson, M., Lundin, S. & Kaller, M. MultiQC: summarize analysis results for multiple tools and samples in a single report. Bioinformatics 32, 3047-3048 (2016).
49 Dobin, A. et al. STAR: ultrafast universal RNA-seq aligner. Bioinformatics 29, 15-21 (2013).
50 Gaspar, J. M. NGmerge: merging paired-end reads via novel empirically-derived models of sequencing errors. BMC Bioinformatics 19, 536 (2018).
51 Patro, R., Duggal, G., Love, M. I., Irizarry, R. A. & Kingsford, C. Salmon provides fast and bias-aware quantification of transcript expression. Nat. Methods 14, 417-419 (2017).
52 Di Tommaso, P. et al. Nextflow enables reproducible computational workflows. Nat. Biotechnol. 35, 316-319 (2017).
53 Gruning, B. et al. Bioconda: sustainable and comprehensive software distribution for the life sciences. Nat. Methods 15, 475-476 (2018).
54 Love, M. I., Huber, W. & Anders, S. Moderated estimation of fold change and dispersion for RNA-seq data with DESeq2. Genome Biol. 15, 550 (2014).
55 Zhu, A., Ibrahim, J. G. & Love, M. I. Heavy-tailed prior distributions for sequence count data: removing the noise and preserving large differences. Bioinformatics 35, 2084-2092 (2019).
56 Storey, J. D. & Tibshirani, R. Statistical significance for genomewide studies. Proc. Natl. Acad. Sci. USA 100, 9440-9445 (2003).
57 Hanzelmann, S., Castelo, R. & Guinney, J. GSVA: gene set variation analysis for microarray and RNA-seq data. BMC Bioinformatics 14, 7 (2013).
58 Stuart, T. et al. Comprehensive integration of single-cell data. Cell 177, 1888-1902.e1821 (2019).
59 Deprez, M. et al. A single-cell atlas of the human healthy airways. Am. J. Respir. Crit. Care Med. 202, 1636-1645 (2020).
60 Travaglini, K. J. et al. A molecular cell atlas of the human lung from single-cell RNA sequencing. Nature 587, 619-625 (2020).
61 Vieira Braga, F. A. et al. A cellular census of human lungs identifies novel cell states in health and in asthma. Nat. Med. 25, 1153-1163 (2019).
62 Bioinformatics and evolutionary genomics. https://bioinformatics.psb.ugent.be/cgi-bin/liste/Venn/calculate_venn.htpl

Sequences

| SEQ ID | | |
|---|---|---|
| NO: | Description | Sequence |
| 1. | tozorakimab VHCDR1 (amino | SYAMS |
| | acid) | |
| 2. | tozorakimab VHCDR2 (amino | GISAIDQSTYYADSVKG |
| | acid) | |
| 3. | tozorakimab VHCDR3 (amino | QKFMQLWGGGLRYPFGY |
| | acid) | |
| 4. | tozorakimab VH Domain (amino | EVQLLESGGGLVQPGGSLRLSCAASGFTFSS |
| | acid) | YAMSWVRQAPGKGLEWVSGISAIDQSTYY |
| | | ADSVKGRFTISRDNSKNTLYLQMNSLRAED |
| | | TAVYYCARQKFMQLWGGGLRYPFGYWGQ |
| | | GTMVTVSS |
| 5. | tozorakimab VLCDR1 (amino | SGEGMGDKYAA |
| | acid) | |
| 6. | tozorakimab VLCDR2 (amino | RDTKRPS |
| | acid) | |
| 7. | tozorakimab VLCDR3 (amino | GVIQDNTGV |
| | acid) | |
| 8. | tozorakimab VL Domain (amino | SYVLTQPPSVSVSPGQTASITCSGEGMGDK |
| | acid) | YAAWYQQKPGQSPVLVIYRDTKRPSGIPER |
| | | FSGSNSGNTATLTISGTQAMDEADYYCGVI |
| | | QDNTGVFGGGTKLTVL |
| 9. | tozorakimab light chain (amino | SYVLTQPPSVSVSPGQTASITCSGEGMGDK |
| | acid) | YAAWYQQKPGQSPVLVIYRDTKRPSGIPER |
| | | FSGSNSGNTATLTISGTQAMDEADYYCGVI |
| | | QDNTGVFGGGTKLTVLGQPKAAPSVTLFPP |
| | | SSEELQANKATLVCLISDFYPGAVTVAWKA |
| | | DSSPVKAGVETTTPSKQSNNKYAASSYLSL |
| | | TPEQWKSHRSYSCQVTHEGSTVEKTVAPTE |
| | | CS |
| 10. | tozorakimab heavy chain (amino | EVQLLESGGGLVQPGGSLRLSCAASGFTFSS |
| | acid) | YAMSWVRQAPGKGLEWVSGISAIDQSTYY |
| | | ADSVKGRFTISRDNSKNTLYLQMNSLRAED |
| | | TAVYYCARQKFMQLWGGGLRYPFGYWGQ |
| | | GTMVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICN |
| | | VNHKPSNTKVDKKVEPKSCDKTHTCPPCPA |
| | | PELLGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVYT |
| | | LPPSRDELTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| 11. | IL-33 full length protein | MKPKMKYSTNKISTAKWKNTASKALCFKL |
| | | GKSQQKAKEVCPMYFMKLRSGLMIKKEAC |
| | | YFRRETTKRPSLKTGRKHKRHLVLAACQQ |
| | | QSTVECFAFGISGVQKYTRALHDSSITGISPI |
| | | TEYLASLSTYNDQSITFALEDESYEIYVEDL |
| | | KKDEKKDKVLLSYYESQHPSNESGDGVDG |
| | | KMLMVTLSPTKDFWLHANNKEHSVELHKC |
| | | EKPLPDQAFFVLHNMHSNCVSFECKTDPGV |
| | | FIGVKDNHLALIKVDSSENLCT |
| | | ENILFKLSET |
| 12. | 10xHis, Avitag and Factor Xa | MHHHHHHHHHHAAGLNDIFEAQKIEWHEA |
| | protease cleavage site | AIEGR |
| 13. | IL-33 (112-270) | SITGISPITEYLASLSTYNDQSITFALEDESYE |
| | | IYVEDLKKDEKKDKVLLSYYESQHPSNESG |
| | | DGVDGKMLMVTLSPTKDFWLHANNKEHS |
| | | VELHKCEKPLPDQAFFVLHNMHSNCVSFEC |
| | | KTDPGVFIGVKDNHLALIKVDSSENLCTENI |
| | | LFKLSET |
| 14. | IL-33^{C>S} | SITGISPITEYLASLSTYNDQSITFALEDESYE |
| | | IYVEDLKKDEKKDKVLLSYYESQHPSNESG |
| | | DGVDGKMLMVTLSPTKDFWLHANNKEHS |
| | | VELHKSEKPLPDQAFFVLHNMHSNSVSFES |
| | | KTDPGVFIGVKDNHLALIKVDSSENLSTENI |
| | | LFKLSET |

1. An IL-33 antagonist for use in a method of treatment reducing or preventing respiratory tract infection in a subject with an IL-33-mediated respiratory disorder.

2. The IL-33 antagonist for use according to claim 1, wherein the IL-33-mediated respiratory disorder is chronic obstructive pulmonary disorder (COPD).

3. The IL-33 antagonist for use according to any preceding claim, wherein the infection is a respiratory tract viral infection or a respiratory tract bacterial infection.

4. The IL-33 antagonist for use according to claim 3, wherein the infection is a respiratory tract viral infection caused by influenza virus (e.g., Influenza virus A, Influenza virus B), respiratory syncytial virus (RSV), adenovirus, metapneumovirus, cytomegalovirus, parainfluenza virus (e.g., hPIV-1, hPIV-2, hPIV-3, hPIV-4), rhinovirus, adenovirus, coxsackie virus, echo virus, corona virus, herpes simplex virus, SARS-coronavirus or smallpox.

5. The IL-33 antagonist for use according to any of claims 1 to 3, wherein the infection is a respiratory tract bacterial infection caused by Chlamydia pneumoniae or Mycoplasma pnuemoniae.

6. The IL-33 antagonist for use according to any preceding claim, wherein the IL-33 antagonist inhibits IL-33^{ox} activity, thereby increasing club cell activity in the airway epithelium.

7. The IL-33 antagonist for use according to any preceding claim, wherein the IL-33 antagonist inhibits IL-33^{ox} activity, thereby increasing total club cell area in the airway epithelium.

8. The IL-33 antagonist for use according to any preceding claim, wherein the IL-33 antagonist inhibits IL-33^{ox} activity, thereby increasing mRNA expression levels in the airway epithelium of one or more markers selected from: SCGBIBA1, BP1FA1, SCGB3A1, WFDC2, MSMB, LTF, SLPI, C3, HLA-DRA, CXCL1, CD74, CXCL17, MDK, TGM2, HLA-DRB1, CXCL8, CXCL2, HLA-DRB5, CX3CL1 and HLA-DPA1.

9. The IL-33 antagonist for use according to any preceding claim, wherein the IL-33 antagonist inhibits IL-33^{ox} activity, thereby increasing mRNA expression levels in the airway epithelium of one or more markers selected from: SCGBIBA1, BP1FA1, SCGB3A1, WFDC2, MSMB and LTF.

10. The IL-33 antagonist for use according claim 9, wherein the one or more markers is selected from SCGB1BA1 and BP1FA1.

11. The IL-33 antagonist for use according to any preceding claim, wherein the IL-33 antagonist inhibits IL-33^{ox} activity, thereby increasing protein expression levels in the airway epithelium of one or more markers selected from: CCSP, SCGB3A1, WFDC2, Beta-microseminoprotein, lactotransferrin, SPLUNC1, secretory leukocyte protease inhibitor (SLPI), Complement C3, HLA-DR alpha chain, C—X—C motif chemokine ligand 1 (CXCL1), Cluster of Differentiation 74 (CD74), C—X—C motif chemokine 17 (CXCL17), midkine (MDK), Protein-glutamine gamma-glutamyltransferase 2 (TGM2), HLA class II histocompatibility antigen, DRB1 beta chain (HLA-DRB1), chemokine (C—X—C motif) ligand 8 (CXCL8), Chemokine (C—X—C motif) ligand 2 (CXCL2), HLA class II histocompatibility antigen, DRB5 beta chain (HLA-DRB5), chemokine (C-X3-C motif) ligand 1 (CX3CL1) and Major histocompatibility complex, class II, DP alpha 1 (HLA-DPA1).

12. The IL-33 antagonist for use according to any preceding claim, wherein the IL-33 antagonist inhibits IL-33^{ox} activity, thereby increasing protein expression levels in the airway epithelium of one or more markers selected from: CCSP, SCGB3A1, WFDC2, Beta-microseminoprotein, lactotransferrin and SPLUNC1.

13. The IL-33 antagonist for use according to claim 12, wherein the one or more markers is selected from CCSP and SPLUNC1.

14. The IL-33 antagonist for use according to any of claims 6 to 13, wherein the airway epithelium comprises the lower airway epithelium.

15. The IL-33 antagonist for use according to claim 14, wherein the lower airway epithelium comprises cuboidal epithelium.

16. The IL-33 antagonist for use according to claim 14, wherein the lower airway epithelium comprises squamous epithelium.

17. The IL-33 antagonist for use according to claims 6 to 13, wherein the airway epithelium comprises upper airway epithelium.

18. The IL-33 antagonist for use according to claim 17, wherein the upper airway epithelium comprises ciliated pseudostratified columnar epithelium.

19. The IL-33 antagonist for use according to any of claims 8 to 18, wherein the mRNA expression level is measured by qRT-PCR.

20. The IL-33 antagonist for use according to claims 11 to 18, wherein the protein expression level is measured by enzyme-linked immunosorbent assay (ELISA), immunohistochemistry (IHC), immunofluorescence, flow cytometry or Western blot.

21. The IL-33 antagonist for use according to any preceding claim, wherein the mRNA expression level or protein expression level is measured within a biological sample obtained from the subject.

22. The IL-33 antagonist for use according to claim 21, wherein the biological sample is selected from a respiratory epithelium biopsy, bronchial brushing, bronchoalveolar fluid (BALF), sputum, serum, plasma or nasal mucosal lining fluid.

23. The IL-33 antagonist for use according to any preceding claim, wherein the IL-33 antagonist inhibits IL-33^{ox} activity, thereby increasing club cell defence function in the airway epithelium.

24. The IL-33 antagonist for use according to claim 23, wherein increasing club cell defence function in the airway epithelium comprises increasing the activity of one or more proteins selected from: CCSP, SCGB3A1, WFDC2, Beta-microseminoprotein, lactotransferrin, SPLUNC1, SLPI, C3, HLA-DRA, CXCL1, CD74, CXCL17, MDK, TGM2, HLA-DRB1, CXCL8, CXCL2, HLA-DRB5, CX3CL1 and HLA-DPA1.

25. The IL-33 antagonist for use according to claim 24, wherein increasing club cell defence function in the airway epithelium comprises increasing the activity of CCSP and/or SPLUNC1.

26. The IL-33 antagonist for use according to any of claims 2 to 25, wherein the method reduces the annualised exacerbation rate in the subject.

27. The IL-33 antagonist for use according to any of claims 2 to 26, wherein the method reduces the frequency of acute exacerbations of COPD (AECOPD) in the subject.

28. The IL-33 antagonist for use according to any preceding claim, wherein the IL-33 antagonist is an IL-33^{ox} antagonist.

29. The IL-33 antagonist for use according to any preceding claim, which is an antibody or antigen binding fragment thereof.

30. The IL-33 antagonist for use according to claim 29, wherein the antibody or antigen binding fragment binds specifically to the reduced form of IL-33 (IL-33red).

31. The IL-33 antagonist for use according to any preceding claim, which is an anti-IL-33 antibody comprising a VH domain comprising HCDR1 having the sequence set forth in SEQ ID NO: 1: HCDR2 having the sequence set forth in SEQ ID NO: 2; and HCDR3 having the sequence set forth in SEQ ID NO: 3; and a VL domain comprising LCDR1 having the sequence set forth in SEQ ID NO: 5; LCDR2 having the sequence set forth in SEQ ID NO: 6 and LCDR3 having the sequence set forth in SEQ ID NO: 7.

32. The IL-33 antagonist for use according to any preceding claim, which is an anti-IL-33 antibody comprising a VH domain having the sequence set forth in SEQ ID NO: 4 and a VL domain having the sequence set forth in SEQ ID NO: 8.

33. The IL-33 antagonist for use according to any preceding claim, wherein reducing respiratory tract infection means reducing the frequency of respiratory tract infection in the subject.

34. The IL-33 antagonist for use according to claim 33, wherein the subject has COPD, the frequency of respiratory tract infection is reduced when the number of AECOPD is statistically lower in a subject over a period of time following the treatment, compared to the number of AECOPD over the same period of time prior to treatment.

35. The IL-33 antagonist for use according to claim 34, the period of time is greater than 6 months, optionally greater than 12 months, optionally between 12 to 24 months, such as 24 months.

36. A composition comprising an IL-33 antagonist for use in a method of treatment preventing or reducing respiratory tract infections in a subject with COPD.

37. The composition for use according to claim 36, wherein the respiratory tract infection is a respiratory tract viral infection.

38. A method for reducing or preventing respiratory tract infection in a subject with COPD comprising administering to said subject a therapeutically effective amount of an IL-33 antagonist.

39. The method of claim 38, wherein the IL-33 antagonist inhibits the activity of oxIL-33.

40. The method of claim 38 or 39, wherein the respiratory tract infection is a respiratory tract viral infection.

41. The method of any of claims 38 to 40, wherein the method reduces the annualised rate of acute exacerbations of COPD (AECOPD) in the subject.

42. An IL-33 antagonist for use in reducing AECOPD in a subject with COPD, wherein the IL-33 antagonist inhibits IL-33^{ox} activity, thereby reducing respiratory tract infections and AECOPD in the subject.

43. The IL-33 antagonist for use according to claim 42, wherein the IL-33 antagonist inhibits IL-33ox activity, thereby increasing total club cell area in the epithelium.

44. The IL-33 antagonist for use according to either of claim 42 or 43, wherein the IL-33 antagonist inhibits IL-33^{ox} activity, thereby increasing mRNA expression levels of one or more markers selected from: SCGBIBA1, BP1FA1, SCGB3A1, WFDC2, MSMB, LTF, SLPI, C3, HLA-DRA, CXCL1, CD74, CXCL17, MDK, TGM2, HLA-DRB1, CXCL8, CXCL2, HLA-DRB5, CX3CL1 and HLA-DPA1.

45. The IL-33 antagonist for use according to any of claims 42 to 44, wherein the IL-33 antagonist inhibits IL-33^{ox} activity, thereby increasing protein expression levels of one or more markers selected from: CCSP, SCGB3A1, WFDC2, Beta-microseminoprotein, lactotransferrin, SPLUNC1, SLPI, C3, HLA-DRA, CXCL1, CD74, CXCL17, MDK, TGM2, HLA-DRB1, CXCL8, CXCL2, HLA-DRB5, CX3CL1 and HLA-DPA1.

46. The IL-33 antagonist for use according to either of claim 44 or 45, wherein the expression level is measured in a biological sample obtained from the subject.

47. The IL-33 antagonist for use according to claim 46, wherein the biological sample is selected from a respiratory epithelium biopsy, bronchial brushing, bronchoalveolar fluid (BALF), sputum, serum, plasma or nasal mucosal lining fluid.

48. Use of an IL-33 antagonist in the manufacture of a medicament for use in a method of treatment preventing of reducing respiratory tract infection in a subject with an IL-33-mediated respiratory disorder.

49. A method of treatment preventing of reducing respiratory tract infection in a subject with an IL-33-mediated respiratory disorder comprising administering a therapeutically effective amount of an IL-33 antagonist to the subject.

50. Use according to claim 48 or method according to claim 49, wherein the method of treatment is as defined in any of claims 1-35.

51. Use according to claim 48 or 50 or method according to claim 49 or 50, wherein the IL-33 antagonist is as defined in any of claims 28 to 32.
